(19) United States
(12) Patent Application Publication
(10) Pub. No.: US 2016/0187315 A1
(43) Pub. Date: Jun. 30, 2016

(54) METHODS FOR DETERMINING CONDITION AND QUALITY OF PETROLEUM PRODUCTS

(71) Applicants: Michael L. Blumenfeld, Haddonfield, NJ (US); James T. Carey, Medford, NJ (US); Gary Christensen, Wenonah, NJ (US); Thomas G. Dietz, Ardmore, PA (US)

(72) Inventors: Michael L. Blumenfeld, Haddonfield, NJ (US); James T. Carey, Medford, NJ (US); Gary Christensen, Wenonah, NJ (US); Thomas G. Dietz, Ardmore, PA (US)

(73) Assignee: ExxonMobil Research and Engineering Company, Annandale, NJ (US)

(21) Appl. No.: 14/976,819

(22) Filed: Dec. 21, 2015

Related U.S. Application Data
(60) Provisional application No. 62/096,564, filed on Dec. 24, 2014.
(60) Provisional application No. 62/096,565, filed on Dec. 24, 2014.

(51) Int. Cl. G01N 33/28

(52) U.S. Cl. CPC G01N 33/2882

(57) ABSTRACT

A method is provided for determining the condition or quality of a product. The method involves adding to the product a taggant in which the taggant exhibits degradation in response to one or more stimuli; carrying out an immunoassay specific for the taggant to determine degradation of the taggant; and determining the condition of the product based on the degradation of the taggant. A method is provided for monitoring degradation or quality of a product. The method involves adding to the product a taggant in which the taggant exhibits degradation in response to one or more stimuli; and carrying out an immunoassay specific for the taggant to determine degradation of the taggant. The method further involves identifying the condition of the product based on the degradation of the taggant. Lubricating engine oils are provided containing the taggant.

### CROSS-REFERENCE TO RELATED APPLICATIONS

This application claims priority to U.S. Provisional Application No. 62/096,564 filed Dec. 24, 2014 and U.S. Provisional Application No. 62/096,565 filed Dec. 24, 2014, which are herein incorporated by reference in their entirety. This application is related to a co-pending U.S. application, filed on an even date herewith, and identified by Attorney Docket No. 2014EM387-US2 (entitled “Methods for Authentication and Identification of Petroleum Products”) which is incorporated herein by reference in its entirety.

### FIELD

This disclosure relates to methods for determining the condition and quality of products (e.g., petroleum products). In particular, this disclosure relates to methods for determining the condition and quality of products (e.g., lubricating engine oils) involving the addition of one or more taggants to the products and an immunoassay specific for the taggants. This disclosure also relates to products containing taggants that exhibit degradation in response to one or more stimuli, and an immunoassay specific for the taggants to determine degradation of the taggants, and then correlating condition of the taggants with condition of the products.

### BACKGROUND

Petroleum products are employed in a variety of automotive, off-highway vehicles, on-highway vehicles, equipment, machines, metal working and industrial applications. It is important to know the identity, quality, condition and remaining useful life of such products to prevent the improper and ineffective utilization of the products. A quality petroleum product insures that the condition of the device/equipment containing the petroleum product is productive and properly functioning. It is, therefore, desirable to monitor the physical and/or chemical conditions and the identity, and the remaining useful life of petroleum products.

Methods exist for the analysis of petroleum products using various reagents in determining the presence and/or concentration of various constituents of the petroleum products. It is generally assumed that as the product ages, these constituents will be consumed, and that if the active constituents persist then the product is assumed fit for service. Specific reagents may be employed for determining the presence and concentration of components in petroleum products. These methods generally analyze for pH, coloring agents, and contaminants using reactive reagents on test strips. These methods generally require controlled conditions. Further, these methods may be subjective and inaccurate and require a detailed knowledge of the product formulation.

Other methods for assessing the quality of a used petroleum product include placing a measured amount of product upon an absorbent material, heating the sample and awaiting dispersion of the sample. The amount of undispersed sample may then be measured and rated quantitatively. These methods and apparatus require significant controlled conditions, including measurement of the product sample volume, the use of a template to measure and rate the quantity of undispersed the sample. Additionally these methods can include heating of the sample, and awaiting dispersal of the sample.

Markers have been used to identify petroleum products. Proton accepting chemical substances, that at a solution concentration of below about 50 milligrams per liter, impart little or no significant color to organic solvents, have been proposed as markers, or taggants, especially for petroleum-derived fuels. The marker is dissolved in a liquid to be identified, and then subsequently detected by performing a chemical test on the marked liquid. Markers are sometimes employed by government agencies to ensure that the appropriate tax has been paid on particular grades of fuel. Oil companies also mark their products to help assist in identifying diluted or altered products. These companies often go to great expense to make sure their branded petroleum products meet certain specifications, for example, volatility and octane number, as well as to provide their petroleum products with effective additive packages containing detergents and other components. Consumers rely upon product names and quality designations to assure that the product being purchased is the quality desired. Thus, it is important to be able to identify a marker in a petroleum product.

Traditionally, the presence of a marker substance is detected and optionally quantified by extracting the fuel with an immiscible aqueous or significantly aqueous solution of an acid substance, the precise nature of which can be varied according to the characteristics of the marker substance. The acid reacts with the basic compound to produce a readily visible, more or less intensely colored cation, that is dissolved in the aqueous acid phase.

The quantity of marker substance in the extract may also be measured, for instance, by visible light absorption spectrophotometry, the results of which are then compared with a reference standard to determine the original concentration of basic marker in the petroleum product. It may be necessary to make repeated, typically two or three, extractions of the product to recover the entire amount of marker originally present in order for complete quantification. Additionally, the measurement requires expensive equipment requiring frequent calibration.

Determining the condition of a used lubricant is challenging. Lubricants of different composition respond differently to aging, contamination, oxidative and thermal abuse. Typical condition monitoring uses electronic sensors that measure either the physical properties of a lubricant (e.g., viscosity, surface tension, color, refractive index) or the change in concentration of contaminant or performance additive elements. The condition of the lubricant must then be inferred from this combination of seemingly ad-hoc measurements.

In advanced cases, a mass selective or spectral technique is used to determine the identity or condition of a lubricant. However, these criteria are impacted in a non-linear fashion, are expensive to run, and require a detailed understanding of the initial lubricant content and behavior. By designing the sensor molecules in an independent fashion, condition monitoring of the equipment can be decoupled from the lubricant chemistry. The lubricant merely transports the sensor molecules throughout the machine environment where condition information and machine health is encoded before detection/analysis.

It would be desirable to have an accurate and easy analytical method to determine the conditions and/or the identity of a petroleum product. It would further be desirable to have an accurate analytical method to determine the petroleum product condition and/or the identity in the field.

A need exists for a simple and rapid method of analyzing a sample of a petroleum product on a qualitative basis to determine condition, origin or other useful property. Also, a need exists for a method to analyze petroleum products rapidly in the field. Further, a need exists for a method to test the quality or the identity of a petroleum product in the field rapidly by untrained personnel and without precision measurement. Still further, a need exists for a diagnostic kit for analysis of petroleum products rapidly in the field.

### SUMMARY

This disclosure relates in part to a method for determining the condition and quality of products (e.g., petroleum products). In particular, this disclosure relates to a method for determining the condition and quality of products (e.g., lubricating engine oils) involving the addition of one or more taggants to the products and an immunoassay specific for the taggants. This disclosure also relates to products containing taggants that exhibit degradation in response to one or more stimuli, and an immunoassay specific for the taggants to determine degradation of the taggants, and then correlating condition of the taggants with condition of the products.

This disclosure also relates in part to a method for determining the condition or quality of a product. The method involves adding to the product a taggant in which the taggant exhibits degradation in response to one or more stimuli; carrying out an immunoassay specific for the taggant to determine degradation of the taggant; and determining the condition of the product based on the degradation of the taggant.

This disclosure further relates in part to a method for monitoring degradation or quality of a product. The method involves adding to the product a taggant in which the taggant exhibits degradation in response to one or more stimuli (e.g., through the use of an industry standardized test); and carrying out an immunoassay specific for the taggant to determine degradation of the taggant. The method further involves identifying the condition of the product based on the degradation of the taggant.

This disclosure yet further relates in part to a method that involves associating a taggant with a product to produce a signature product in which the taggant exhibits degradation in response to one or more stimuli; identifying the taggant in the signature product by an immunoassay specific for the taggant; mapping the taggant of the signature product to a product code or a batch code of the signature product; obtaining a test product to determine the condition and/or identity of the test product; identifying the presence or absence of a taggant in the test product by an immunoassay specific for the taggant; and comparing results of the immunoassay carried out on the test product with results of the immunoassay carried out on the signature product to determine the condition, quality, identity and/or remaining useful life of the test product.

This disclosure also relates in part to a lubricating engine oil having a composition that includes a lubricating oil base stock as a major component; and a taggant, as a minor component. The taggant exhibits degradation in response to one or more stimuli. The taggant is present in an amount sufficient for an immunoassay to be carried out specific for the taggant to determine degradation of the taggant.

This disclosure further relates in part to a diagnostic kit for the analysis of products. The kit includes one or more immunoassay interrogation devices, one or more immunoassay test strips, and immunoassay instructions.

It has been surprisingly found that, in accordance with this disclosure, taggants can be used in immunoassay methods for determining the condition and/or identity of lubricating oils. The immunoassay methods can be used, particularly in the field, for determining the condition and/or identity of lubricating oils. In particular, it has been surprisingly found that, in accordance with this disclosure, taggants that exhibit degradation in response to one or more stimuli show unique benefits when used as taggants in conjunction with immunoassay methods for determining the condition and/or identity of lubricating oils.

Further, it has been surprisingly found that amide taggants show particular advantages when used in lubricating oils due to their thermal stability, oxidative stability, moderate degradation kinetics, solubility and compatibility with additives used in lubricating oil compositions. The performance of amide compounds is set apart from other taggants used in conjunction with immunoassays, such as proteins or nitrogen containing small molecules, that do not contain amide functionality. The immunoassay methods can be used, particularly in the field, for determining the condition and/or identity of lubricating oils.

Other objects and advantages of the present disclosure will become apparent from the detailed description that follows.

### BRIEF DESCRIPTION OF THE DRAWINGS

FIG. 1 shows examples of “detect” and “no detect” from immunoassay interrogation of a lubricating oil using a taggant in accordance with this disclosure.

FIG. 2 graphically shows the use of taggants to detect oxidation in accordance with this disclosure.

FIG. 3 shows examples of tag molecules that can be used in lubricants and their utility in determining the condition and/or identity of lubricating oils in accordance with this disclosure.

FIG. 4 shows examples of detection from immunoassay interrogation of a lubricating oil using a taggant (i.e., screening for initial concentration) in accordance with step 3 of the procedure for immunoassay for condition monitoring in the Examples.

FIG. 5 shows examples of detection from immunoassay interrogation of a lubricating oil using a taggant (i.e., use of immunoassay to monitor lubricant condition) in accordance with step 4 of the procedure for immunoassay for condition monitoring in the Examples.

### DETAILED DESCRIPTION

All numerical values within the detailed description and the claims herein are modified by “about” or “approximately” the indicated value, and take into account experimental error and variations that would be expected by a person having ordinary skill in the art.

In general, this disclosure involves chemical cryptography, in particular, the use of an inert marker or a variable array of inert markers for the purpose of establishing the condition and/or identity of a petroleum product, e.g., lubricating oil, by immunoassay methods.

This disclosure uses molecular tags to correlate the condition of petroleum products (e.g., lubricants) in-service with bench-top screening tests used in lubricant development. A sensor molecule (or multiple sensor molecules called an “array”) is placed into the lubricating oil at manufacture or aftermarket through the use of a sensor “spike”.

These molecules are detectable using a standard, semi-quantitative analytical technique such as lateral flow immunoassay. The initial concentrations of the sensor molecules are chosen such that they are detectable in fresh oil, but undetectable after being subjected to stressful conditions (e.g., 600 hr RPVOT ASTM D2272). In this manner, a customer can be informed that absence of a chemical sensor in service implies that the lubricating oil has been subjected to environmental stress equivalent to or greater than a given bench screener.

Molecular sensors can also be identified to indicate contamination from another lubricant, contact with acidic or caustic conditions, thermal excursions or reductive/oxidative conditions. Detection of these molecules can then be multiplexed for a detailed history and condition of a used lubricant, with clear correlation to bench screeners used in development. As the molecules are at inert concentrations (e.g., <1 ppm), they do not impact performance and a large (e.g., 20 or more sensor molecules) basis set can be established.

This disclosure improves over current state of the art, whereby concentrations of performance additives and contaminants or physical properties of the petroleum product (e.g., lubricant) are the only measurements used in condition monitoring and these cannot be measured without advanced lab equipment. Lubricant additives are not designed to respond to specific stimuli or a specific history. Additionally, the number of lubricant additives is typically 10, and these are chosen primarily for lubrication performance features. The small number of additives limits the number of dimensions the analytical query can investigate (e.g., concentration of each of the 10 lubricant additives). Also, lubricant physical properties are impacted in non-linear fashion and cannot be traced back to a single influencer (e.g., both oxidative decomposition and contamination can impact lubricant surface tension, however, a molecular sensor chosen for oxidative response will not be impacted by contamination).

In particular, this disclosure uses a taggant or an array of taggants that exhibit degradation in response to one or more stimuli (e.g., acidic conditions, basic or caustic conditions, thermal excursions, reductive/oxidative conditions, photochemical conditions, and contamination from another source) to determine the condition and/or identity of lubricating oils. The taggant array is a series of N (where N=1 or more) taggant molecules, each with a separate chemical identity that can be independently detected (e.g. by immunoassay) as either “present” or “absent” in the lubricant product. Each of the taggants in the array can either be included or omitted in a given batch of lubricant at manufacture, providing 2^{N} unique combinations in the array.

In general, the disclosure features a method of marking a petroleum product for monitoring condition and history thereof in which a marker, composed of a taggant (e.g., one or more amide compounds) or a taggant array (e.g., two or more amide compounds) is associated with the petroleum product. The marker is non-deleterious to the petroleum product, is used sparingly in the petroleum product, is soluble in water, does not interact with the petroleum product chemistry, and is robustly detectable. The presence of the marker can only be easily established by someone who knows the identity of the marker, but cannot be routinely determined by a person unfamiliar with the marker. The markers exhibit degradation in response to one or more stimuli (e.g., acidic conditions, basic or caustic conditions, thermal excursions, reductive/oxidative conditions, photochemical conditions, and contamination from another source).

As used herein, by marker is meant a taggant that comprises one or more compounds, preferably one or more amide compounds. More preferably, the marker is a taggant array that comprises two or more amide compounds chosen for their clear response to relevant stimuli in service.

As used herein, by marking a product for monitoring condition and history thereof is meant associating a marker with a product so that the condition, source, identity, or other information about the product may be established. Identification of a marked product can also facilitate: 1) authenticating the product; 2) monitoring of manufacturing or other processes, including monitoring process streams and blending controls; 3) product monitoring for security or regulatory purposes, such as marking the source country of products for customs and marking regulated substances; 4) detecting and monitoring spillages of marked materials, including the detection of residues of marked products, such as toxic wastes, organic pollutants and other chemicals; 5) tracing a product, such as marking a process chemical to monitor the rate of addition of the chemical to a system in order to optimize chemical dosage; and 6) studies of biodegradation of a compound, e.g., in soil biodegradation studies. Marking a product for monitoring condition and history thereof also includes the associating a product with a particular concentration of a marker, so to facilitate the detection of product adulteration by way of dilution, concentration changes, or the addition of foreign substances.

In accordance with this disclosure, a method for determining the condition of a product is provided. The method includes adding to the product a taggant (e.g., a taggant array) in which the taggant exhibits degradation in response to one or more stimuli (e.g., acidic conditions, basic or caustic conditions, thermal excursions, reductive/oxidative conditions, photochemical conditions, and contamination from another source); carrying out an immunoassay specific for the taggant to determine degradation of the taggant; and determining the condition of the product based on the degradation of the taggant.

The taggant (e.g., taggant array) of this disclosure is capable of being detected by immunoassay. The immunoassay is carried out using a test strip that is specific for the taggant. The test strip can preferably be a lateral flow immunoassay.

Also, in accordance with this disclosure, a method for monitoring degradation of a product is provided. The method includes adding to the product a taggant (e.g., a taggant array) in which wherein the taggant exhibits degradation in response to one or more stimuli (e.g., acidic conditions, basic or caustic conditions, thermal excursions, reductive/oxidative conditions, photochemical conditions, and contamination from another source); and carrying out an immunoassay specific for the taggant to determine degradation of the taggant.

The taggant (e.g., taggant array) of this disclosure is capable of being detected by immunoassay. The immunoassay is carried out using a test strip that is specific for the taggant. The test strip can preferably be a lateral flow immunoassay.

Further, in accordance with this disclosure, a method is provided that associates a taggant (e.g., a taggant array) with a product to produce a signature (i.e., genuine) product. The taggant exhibits degradation in response to one or more stimuli. The method also identifies the taggant in the signature product by an immunoassay specific for the taggant; maps the taggant of the signature product to a product code or a batch code of the signature product; obtains a test product to determine the condition and/or identity of the test product; identifies the presence or absence of a taggant in the test product by an immunoassay specific for the taggant; and compares results of the immunoassay carried out on the test product with results of the immunoassay carried out on the signature product to determine the condition and/or identity of the test product.

In an embodiment, the method maps the taggant (e.g., taggant array) of the signature product to a product code or a batch code of the signature product through the use of a decoder key. The mapped taggant of the signature product to a product code or a batch code of the signature product is preferably obtained from a supplier website or database. The mapped taggant of the signature product to a product code or a batch code of the signature product is then compared with an immunoassay carried out on a purchased product to determine the condition and/or identity of the purchased product.

The taggant (e.g., taggant array) of this disclosure is capable of being detected by immunoassay. The immunoassay is carried out using a test strip that is specific for the taggant. The test strip can be a coded test strip that can be read by a bar code reader.

In an embodiment, the test strip can include a taggant and a product identification (e.g., a taggant array and a product identification array). The test strip can preferably be a lateral flow immunoassay.

The taggants and taggant arrays useful in this disclosure exhibit degradation in response to one or more stimuli (e.g., acidic conditions, basic or caustic conditions, thermal excursions, reductive/oxidative conditions, photochemical conditions, and contamination from another source). The detection of degradation for a particular stimuli allows one to determine the condition and/or identity of petroleum products (e.g., lubricating oils) with respect to the particular stimuli.

In accordance with this disclosure, a lubricating engine oil is provided having a composition comprising a lubricating oil base stock as a major component; and a taggant (e.g., a taggant array that comprises two or more amide compounds), as a minor component. The taggant exhibits degradation in response to one or more stimuli (e.g., acidic conditions, basic or caustic conditions, thermal excursions, reductive/oxidative conditions, photochemical conditions, and contamination from another source). The taggant is present in an amount sufficient for an immunoassay to be carried out specific for the taggant to determine degradation of the taggant.

Illustrative taggants useful in this disclosure include, for example, amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, and the like.

As indicated herein, the taggant can comprise one or more amide compounds (e.g., a taggant array that comprises two or more amide compounds). In particular, the taggant compounds are selected from aliphatic amide compounds and/or cyclic amide compounds. The one or more amide compounds include (i) one or more aliphatic amide compounds, (ii) one or more cyclic amide compounds, or (iii) a mixture of at least one aliphatic amide compound and at least one cyclic amide compound.

The aliphatic amide compounds include, for example, pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, N-[2-(1H-indol-3-yl)ethyl]heptadecanamide, and the like.

The cyclic amide compounds include, for example, (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1, 2-benzisoxazol-3-yl)-1-piperidinyl]ethyl}-2-methyl-6, 7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), tert-butyl 2,4-dioxo-1-piperidinecarboxylate, and the like.

Illustrative aminic compounds include, for example, diphenylamines, alkylated diphenyl amines, alkylnaphthyl amines, alkyl aryl naphthyl amines, alkyl aryl amines, polyethylene amines, dialkyl amines, polyisobutene amines, polypropylene amines, and the like.

Illustrative aromatic compounds include, for example, biphenyls, benzoates, carbazoles, polycyclic aromatic compounds, nitrobenzenes, diphenyl amines, phenylhydrazines, diphenyl ethers, toluenes, xylenes, pyrenes, pyrroles, furans, pyridines, and the like.

Illustrative phenolic compounds include, for example, simple phenols, phenolic acis, benzoquinones, phenolic aldehydes, xanthonoids, naphthoquinones, flavonoids, bioflavonoids, lignins, polyphenols alkylphenols, sulfurized alkylphenols, t-butyl-4-heptyl phenol, and the like.

Illustrative sulfur-containing compounds include, for example, bisulfites, dithionates, dithionites, glucosinolates, sulfides, sulfites, thocyanates, thionyl compounds, persulfates, phosphorothioates, thioureas, metabisulfites, thiols, alkyl thiols, sulfur oxoacids, organic sulfides, zinc dithiophosphates, and the like.

Illustrative heterocyclic compounds include, for example, furans, tetrahydrofurans, thiophenes, pyrroles, pyrrolidines, pyrans, pyridines, piperidines, imidazoles, thiazoles, dioxanes, morpholines, indoles, isoindoles, indolizines, quinolones, isoquinolines, purines carbazoles, dibenzofurans, xanthenes pyrimidines, and the like.

Illustrative ester compounds include, for example, monoesters, di-esters, polyol esters, complex esters, organic esters, acetates, formates, butyrates, benzoates, inorganic esters, and the like.

Illustrative carboxylic acid compounds include, for example, pentanoic acids (C5) caprylic acids (C8), pelargonic acids (C9), capric acids (C10), undecylic acids (C11), lauric acids (C12), tridecylic acids (C13), myristic acids (C14), pentadecylic acids (C15), palmitic acids (C16), margaric acids (C17), stearic acids (C18), nonadecylic acids (C19), arachidic acids (C20), heneicosylic acids (C21), behenic acids (C22), tricosylic acids (C23), lignoceric acids (C24), pentacosylic acids (C25), cerotic acids (C26), and the like.

Illustrative aldehyde compounds include, for example, butyraldehyde, benzaldehydes, cinnamaldehydes, tolualdehydes, furfurals, retinaldehydes, glyoxals, succindialdehydes, glutaraldehydes, lactaldehydes, phthalaldehydes, fatty aldehydes, and the like.

Illustrative ketone compounds include, for example, acetophenones, alkyl acetophenones, benzophenones, fructose, cyclopentanones, benzo cyclopentanones, cyclohexanones, alkyl cyclohexanones, alkyl ketones, and the like.

Illustrative alcohol compounds include, for example, cyclohexanols, phenols, glycols, polyols, sugar alcohols, hydroxybutyric acids, fatty alcohols, to and the like.

Illustrative imide compounds include, for example, n-ethylmaleimide, phthalimide, captan, cycloheximide, and the like.

Illustrative acidic compounds include, for example, caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), and mixtures thereof, and the like.

Illustrative basic compounds include, for example, diphenylamines, alkylated diphenyl amines, alkylnaphthyl amines, alkyl aryl naphthyl amines, alkyl aryl amines, polyethylene amines, dialkyl amines, polyisobutene amines, polypropylene amines, phenols, and the like.

Illustrative compounds sensitive to oxidative conditions include, for example, metal bipyridines, nitrophenanthrolines, N-phenylanthranilic acids, ferroins, n-ethoxychroidine, dianisidines, diphenylamine sulfonates, diphenylbenzidines, diphenylamines, viologens, idophenols, thionines, phenosafranins, indigomono sulfonic acids, and the like.

Illustrative compounds sensitive to reductive conditions include, for example, metal bipyridines, nitrophenanthrolines, N-phenylanthranilic acids, ferroins, n-ethoxychroidine, dianisidines, diphenylamine sulfonates, diphenylbenzidines, diphenylamines, viologens, idophenols, thionines, phenosafranins, indigomono sulfonic acids, and the like.

Illustrative thermally labile compounds include, for example, zinc dithiophosphates, asphaltenes, polyisobutenes, ethylene propylene copolymers, proteins, and the like.

Illustrative yellow-metal active compounds include, for example, benzotriazoles, triazoles, 2-mercaptobenzothiazoles, tolyltriazoles, and the like.

Illustrative volatile compounds include, for example, low molecular weight esters, amides, alcohols, ketones, aldehydes, amines, and the like.

Illustrative hydrolytically unstable compounds include, for example, monoesters, diesters, polyethylene glycols, and the like.

Illustrative surface active compounds include, for example, zinc dithiophosphates, metal phenolates, basic metal sulfonates, fatty acids, amines triazoles, benzotriazoles, 2-mercapto benzothiazoles, tolytriazoles, and the like.

Illustrative contaminant scavenging compounds include, for example, N,N′-disalicylidene-1,2-diaminopropane, succinimides, alkylene succinimides, alkyl acetoacetates, imidazolidines, and the like.

Illustrative elastomer partitioning additives include, for example, organic phosphates, aromatic esters, aromatic hydrocarbons, esters (butylbenzyl phthalate, for example), polybutenyl succinic anhydrides, and the like.

Illustrative oxygen sensitive compounds include, for example, metal bipyridines, nitrophenanthrolines, N-phenylanthranilic acids, ferroins, n-ethoxychroidine, dianisidines, diphenylamine sulfonates, diphenylbenzidines, diphenylamines, viologens, idophenols, thionines, phenosafranins, indigomono sulfonic acids, and the like.

Illustrative light sensitive compounds include, for example, coumarins, quinones, sinapinic acid esters, alpha-cyano-4-hydroxycinnamic acid esters, alkylated ditbranols, alkylated trihydroxyacetophenones, trans-3-indoleacrylic acid esters, ferulic acid esters, picolinic acid esters, alkylated versions of 6-aza-2-thiothymine, and the like.

The taggant (e.g., taggant array) is present in an amount of from about 0.05 ppm to about 20 ppm, preferably from about 0.1 ppm to about 10 ppm, and more preferably from about 0.2 ppm to about 5 ppm.

The lubricating oil base stock preferably comprises a Group I, Group II, Group III, Group IV, or Group V base oil. The lubricating oil base stock is present in an amount of from about 70 weight percent to about 95 weight percent, based on the total weight of the lubricating engine oil.

The lubricating engine oil can further include one or more of an antiwear additive, viscosity modifiers, antioxidant, detergent, dispersant, pour point depressant, corrosion inhibitor, metal deactivator, seal compatibility additive, anti-foam agent, inhibitor, and anti-rust additive.

In accordance with this disclosure, a kit is provided for monitoring the condition and/or identifying the source of a petroleum product by the methods described herein. The kit includes one or more test strips for carrying out immunoassay, preferably lateral flow immunoassay, of petroleum products. The test strips are specific for a taggant (e.g., taggant array). The test strips can be coded test strips that can be read by a bar code reader. The test strips can also include a taggant and a product identification (e.g., a taggant array and a product identification array).

The kits of this disclosure may also include a documents for comparing the result of the detection assay with that expected from a genuine product, and may comprise instructions describing the result expected of a genuine product. The documents may include, for example, a color chart, calibration table or calibration curve. The kit may comprise a sample of marked material identical to marked genuine product to be analyzed alongside the unknown sample.

The ability to provide assay testing in kit form ensures that a person in the field, such as a supplier of a product in an environment distant from the product source, can quickly check the condition and/or identity of the product without recourse to laboratory facilities.

Illustrative petroleum products useful in this disclosure include, for example, lubricating oils, automatic transmission fluids, engine oils, traction drive transmission fluids, manual transmission fluids, power steering fluids, antifreeze fluids, greases, crankcase lubricants, mineral oils, oils with Group 1, 2, 3 or 4 base oils, differential lubricants, turbine lubricants, gear lubricants, gear box lubricants, axle lubricants, brake fluids, farm tractor fluids, transformer fluids, compressor fluids, cooling system fluids, metal working fluids, hydraulic fluids, industrial fluids, fuels, continuously variable transmission fluid, infinitely variable transmission fluids, and mixtures thereof.

In particular, petroleum products useful in this disclosure can include, for example, lubricating oils, gasoline, diesel fuel, biodiesel fuel, kerosene, and industrial solvents, such as ethanol, hexane, toluene, xylenes, naptha, aromatic solvents (100, 150, 200, etc.), aliphatic solvents (C6, C9, etc.), mineral oil, and the like.

### Lubricating Oil Base Stocks

A wide range of lubricating base oils is known in the art. Lubricating base oils that are useful in the present disclosure are natural oils, mineral oils and synthetic oils, and unconventional oils (or mixtures thereof) can be used unrefined, refined, or rerefined (the latter is also known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process.

Refined oils are similar to the oils discussed for unrefined oils except refined oils are subjected to one or more purification steps to improve at least one lubricating oil property. One skilled in the art is familiar with many purification processes. These processes include solvent extraction, secondary distillation, acid extraction, base extraction, filtration, and percolation. Rerefined oils are obtained by processes analogous to refined oils but using an oil that has been previously used as a feed stock.

Groups I, II, III, IV and V are broad base oil stock categories developed and defined by the American Petroleum Institute (API Publication 1509; www.API.org) to create guidelines for lubricant base oils. Group I base stocks have a viscosity index of between about 80 to 120 and contain greater than about 0.03% sulfur and/or less than about 90% saturates. Group II base stocks have a viscosity index of between about 80 to 120, and contain less than or equal to about 0.03% sulfur and greater than or equal to about 90% saturates.

Group III stocks have a viscosity index greater than about 120 and contain less than or equal to about 0.03% sulfur and greater than about 90% saturates.

Group IV includes polyalphaolefins (PAO). Group V base stock includes base stocks not included in Groups I-IV. The table below summarizes properties of each of these five groups.

| | Base Oil Properties | | |
|---|---|---|---|
| | Saturates | Sulfur | Viscosity Index |
| Group I | <90 and/or | >0.03% and | ≧80 and <120 |
| Group II | ≧90 and | ≦0.03% and | ≧80 and <120 |
| Group III | ≧90 and | ≦0.03% and | ≧120 |

| | |
|---|---|
| Group IV | polyalphaolefins (PAO) |
| Group V | All other base oil stocks not included in Groups I, II, III or IV |

Natural oils include animal oils, vegetable oils (castor oil and lard oil, for example), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful. Natural oils vary also as to the method used for their production and purification, for example, their distillation range and whether they are straight run or cracked, hydrorefined, or solvent extracted.

Group II and/or Group III hydroprocessed or hydrocracked base stocks, including synthetic oils such as alkyl aromatics and synthetic esters are also well known base stock oils.

Synthetic oils include hydrocarbon oil. Hydrocarbon oils include oils such as polymerized and interpolymerized olefins (polybutylenes, polypropylenes, propylene isobutylene copolymers, ethylene-olefin copolymers, and ethylene-alphaolefin copolymers, for example). Polyalphaolefin (PAO) oil base stocks are commonly used synthetic hydrocarbon oil. By way of example, PAOs derived from C₈, C₁₀, C₁₂, C₁₄ olefins or mixtures thereof may be utilized. See U.S. Pat. Nos. 4,956,122; 4,827,064; and 4,827,073.

The number average molecular weights of the PAOs, which are known materials and generally available on a major commercial scale from suppliers such as ExxonMobil Chemical Company, Chevron Phillips Chemical Company, BP, and others, typically vary from about 250 to about 3,000, although PAO's may be made in viscosities up to about 150 cSt (100° C.). The PAOs are typically comprised of relatively low molecular weight hydrogenated polymers or oligomers of alphaolefins which include, but are not limited to, C₂ to about C₃₂ alphaolefins with the C₈ to about C₁₆ alphaolefins, such as 1-octene, 1-decene, 1-dodecene and the like, being preferred. The preferred polyalphaolefins are poly-1-octene, poly-1-decene and poly-1-dodecene and mixtures thereof and mixed olefin-derived polyolefins. However, the dimers of higher olefins in the range of C₁₄ to C₁₈ may be used to provide low viscosity base stocks of acceptably low volatility. Depending on the viscosity grade and the starting oligomer, the PAOs may be predominantly trimers and tetramers of the starting olefins, with minor amounts of the higher oligomers, having a viscosity range of 1.5 to 12 cSt. PAO fluids of particular use may include 3.0 cSt, 3.4 cSt, and/or 3.6 cSt and combinations thereof. Mixtures of PAO fluids having a viscosity range of 1.5 to approximately 150 cSt or more may be used if desired.

The PAO fluids may be conveniently made by the polymerization of an alphaolefin in the presence of a polymerization catalyst such as the Friedel-Crafts catalysts including, for example, aluminum trichloride, boron trifluoride or complexes of boron trifluoride with water, alcohols such as ethanol, propanol or butanol, carboxylic acids or esters such as ethyl acetate or ethyl propionate. For example the methods disclosed by U.S. Pat. No. 4,149,178 or 3,382,291 may be conveniently used herein. Other descriptions of PAO synthesis are found in the following U.S. Pat. Nos. 3,742,082; 3,769,363; 3,876,720; 4,239,930; 4,367,352; 4,413,156; 4,434,408; 4,910,355; 4,956,122; and 5,068,487. The dimers of the C₁₄ to C₁₈ olefins are described in U.S. Pat. No. 4,218,330.

Other useful lubricant oil base stocks include wax isomerate base stocks and base oils, comprising hydroisomerized waxy stocks (e.g. waxy stocks such as gas oils, slack waxes, fuels hydrocracker bottoms, etc.), hydroisomerized Fischer-Tropsch waxes, Gas-to-Liquids (GTL) base stocks and base oils, and other wax isomerate hydroisomerized base stocks and base oils, or mixtures thereof. Fischer-Tropsch waxes, the high boiling point residues of Fischer-Tropsch synthesis, are highly paraffinic hydrocarbons with very low sulfur content. The hydroprocessing used for the production of such base stocks may use an amorphous hydrocracking/hydroisomerization catalyst, such as one of the specialized lube hydrocracking (LHDC) catalysts or a crystalline hydrocracking/hydroisomerization catalyst, preferably a zeolitic catalyst. For example, one useful catalyst is ZSM-48 as described in U.S. Pat. No. 5,075,269, the disclosure of which is incorporated herein by reference in its entirety. Processes for making hydrocracked/hydroisomerized distillates and hydrocracked/hydroisomerized waxes are described, for example, in U.S. Pat. Nos. 2,817,693; 4,975,177; 4,921,594 and 4,897,178 as well as in British Patent Nos. 1,429,494; 1,350,257; 1,440,230 and 1,390,359. Each of the aforementioned patents is incorporated herein in their entirety. Particularly favorable processes are described in European Patent Application Nos. 464546 and 464547, also incorporated herein by reference. Processes using Fischer-Tropsch wax feeds are described in U.S. Pat. Nos. 4,594,172 and 4,943,672, the disclosures of which are incorporated herein by reference in their entirety.

Gas-to-Liquids (GTL) base oils, Fischer-Tropsch wax derived base oils, and other wax-derived hydroisomerized (wax isomerate) base oils be advantageously used in the instant disclosure, and may have useful kinematic viscosities at 100° C. of about 3 cSt to about 50 cSt, preferably about 3 cSt to about 30 cSt, more preferably about 3.5 cSt to about 25 cSt, as exemplified by GTL 4 with kinematic viscosity of about 4.0 cSt at 100° C. and a viscosity index of about 141. These Gas-to-Liquids (GTL) base oils, Fischer-Tropsch wax derived base oils, and other wax-derived hydroisomerized base oils may have useful pour points of about −20° C. or lower, and under some conditions may have advantageous pour points of about −25° C. or lower, with useful pour points of about −30° C. to about −40° C. or lower. Useful compositions of Gas-to-Liquids (GTL) base oils, Fischer-Tropsch wax derived base oils, and wax-derived hydroisomerized base oils are recited in U.S. Pat. Nos. 6,080,301; 6,090,989, and 6,165,949 for example, and are incorporated herein in their entirety by reference.

The hydrocarbyl aromatics can be used as a base oil or base oil component and can be any hydrocarbyl molecule that contains at least about 5% of its weight derived from an aromatic moiety such as a benzenoid moiety or naphthenoid moiety, or their derivatives. These hydrocarbyl aromatics include alkyl benzenes, alkyl naphthalenes, alkyl diphenyl oxides, alkyl naphthols, alkyl diphenyl sulfides, alkylated bis-phenol A, alkylated thiodiphenol, and the like. The aromatic can be mono-alkylated, dialkylated, polyalkylated, and the like. The aromatic can be mono- or poly-functionalized. The hydrocarbyl groups can also be comprised of mixtures of alkyl groups, alkenyl groups, alkynyl, cycloalkyl groups, cycloalkenyl groups and other related hydrocarbyl groups. The hydrocarbyl groups can range from about C₆ up to about C₆₀ with a range of about C₈ to about C₂₀ often being preferred. A mixture of hydrocarbyl groups is often preferred, and up to about three such substituents may be present. The hydrocarbyl group can optionally contain sulfur, oxygen, and/or nitrogen containing substituents. The aromatic group can also be derived from natural (petroleum) sources, provided at least about 5% of the molecule is comprised of an above-type aromatic moiety. Viscosities at 100° C. of approximately 3 cSt to about 50 cSt are preferred, with viscosities of approximately 3.4 cSt to about 20 cSt often being more preferred for the hydrocarbyl aromatic component. In one embodiment, an alkyl naphthalene where the alkyl group is primarily comprised of 1-hexadecene is used. Other alkylates of aromatics can be advantageously used. Naphthalene or methyl naphthalene, for example, can be alkylated with olefins such as octene, decene, dodecene, tetradecene or higher, mixtures of similar olefins, and the like. Useful concentrations of hydrocarbyl aromatic in a lubricant oil composition can be about 2% to about 25%, preferably about 4% to about 20%, and more preferably about 4% to about 15%, depending on the application.

Alkylated aromatics such as the hydrocarbyl aromatics of the present disclosure may be produced by well-known Friedel-Crafts alkylation of aromatic compounds. See Friedel-Crafts and Related Reactions, Olah, G. A. (ed.), Inter-science Publishers, New York, 1963. For example, an aromatic compound, such as benzene or naphthalene, is alkylated by an olefin, alkyl halide or alcohol in the presence of a Friedel-Crafts catalyst. See Friedel-Crafts and Related Reactions, Vol. 2, part 1, chapters 14, 17, and 18, See Olah, G. A. (ed.), Inter-science Publishers, New York, 1964. Many homogeneous or heterogeneous, solid catalysts are known to one skilled in the art. The choice of catalyst depends on the reactivity of the starting materials and product quality requirements. For example, strong acids such as AlCl₃, BF₃, or HF may be used. In some cases, milder catalysts such as FeCl₃ or SnCl₄ are preferred. Newer alkylation technology uses zeolites or solid super acids.

Esters comprise a useful base stock. Additive solvency and seal compatibility characteristics may be secured by the use of esters such as the esters of dibasic acids with monoalkanols and the polyol esters of monocarboxylic acids. Esters of the former type include, for example, the esters of dicarboxylic acids such as phthalic acid, succinic acid, alkyl succinic acid, alkenyl succinic acid, maleic acid, azelaic acid, suberic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc.

Particularly useful synthetic esters are those which are obtained by reacting one or more polyhydric alcohols, preferably the hindered polyols (such as the neopentyl polyols, e.g., neopentyl glycol, trimethylol ethane, 2-methyl-2-propyl-1,3-propanediol, trimethylol propane, pentaerythritol and dipentaerythritol) with alkanoic acids containing at least about 4 carbon atoms, preferably C₅ to C₃₀ acids such as saturated straight chain fatty acids including caprylic acid, capric acid, lauric acid, myristic acid, palmitic acid, stearic acid, arachic acid, and behenic acid, or the corresponding branched chain fatty acids or unsaturated fatty acids such as oleic acid, or mixtures of any of these materials.

Suitable synthetic ester components include the esters of trimethylol propane, trimethylol butane, trimethylol ethane, pentaerythritol and/or dipentaerythritol with one or more monocarboxylic acids containing from about 5 to about 10 carbon atoms. These esters are widely available commercially, for example, the Mobil P-41 and P-51 esters of ExxonMobil Chemical Company.

Also useful are esters derived from renewable material such as coconut, palm, rapeseed, soy, sunflower and the like. These esters may be monoesters, di-esters, polyol esters, complex esters, or mixtures thereof. These esters are widely available commercially, for example, the Mobil P-51 ester of ExxonMobil Chemical Company.

Engine oil formulations containing renewable esters are included in this disclosure. For such formulations, the renewable content of the ester is typically greater than about 70 weight percent, preferably more than about 80 weight percent and most preferably more than about 90 weight percent.

Other useful fluids of lubricating viscosity include non-conventional or unconventional base stocks that have been processed, preferably catalytically, or synthesized to provide high performance lubrication characteristics.

Non-conventional or unconventional base stocks/base oils include one or more of a mixture of base stock(s) derived from one or more Gas-to-Liquids (GTL) materials, as well as isomerate/isodewaxate base stock(s) derived from natural wax or waxy feeds, mineral and or non-mineral oil waxy feed stocks such as slack waxes, natural waxes, and waxy stocks such as gas oils, waxy fuels hydrocracker bottoms, waxy raffinate, hydrocrackate, thermal crackates, or other mineral, mineral oil, or even non-petroleum oil derived waxy materials such as waxy materials received from coal liquefaction or shale oil, and mixtures of such base stocks.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of lubricating viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include oils boiling in the lube oil boiling range (1) separated/fractionated from synthesized GTL materials such as, for example, by distillation and subsequently subjected to a final wax processing step which involves either or both of a catalytic dewaxing process, or a solvent dewaxing process, to produce lube oils of reduced/low pour point; (2) synthesized wax isomerates, comprising, for example, hydrodewaxed or hydroisomerized cat and/or solvent dewaxed synthesized wax or waxy hydrocarbons; (3) hydrodewaxed or hydroisomerized cat and/or solvent dewaxed Fischer-Tropsch (F-T) material (i.e., hydrocarbons, waxy hydrocarbons, waxes and possible analogous oxygenates); preferably hydrodewaxed or hydroisomerized/followed by cat and/or solvent dewaxing dewaxed F-T waxy hydrocarbons, or hydrodewaxed or hydroisomerized/followed by cat (or solvent) dewaxing dewaxed, F-T waxes, or mixtures thereof.

GTL base stock(s) and/or base oil(s) derived from GTL materials, especially, hydrodewaxed or hydroisomerized/followed by cat and/or solvent dewaxed wax or waxy feed, preferably F-T material derived base stock(s) and/or base oil(s), are characterized typically as having kinematic viscosities at 100° C. of from about 2 mm²/s to about 50 mm²/s (ASTM D445). They are further characterized typically as having pour points of −5° C. to about −40° C. or lower (ASTM D97). They are also characterized typically as having viscosity indices of about 80 to about 140 or greater (ASTM D2270).

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than about 10 ppm, and more typically less than about ppm of each of these elements. The sulfur and nitrogen content of GTL base stock(s) and/or base oil(s) obtained from F-T material, especially F-T wax, is essentially nil. In addition, the absence of phosphorous and aromatics make this materially especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

The GTL material, from which the GTL base stock(s) and/or base oil(s) is/are derived is preferably an F-T material (i.e., hydrocarbons, waxy hydrocarbons, wax).

Base oils for use in the formulated lubricating oils useful in the present disclosure are any of the variety of oils corresponding to API Group I, Group II, Group III, Group IV, and Group V oils and mixtures thereof, preferably API Group II, Group III, Group IV, and Group V oils and mixtures thereof, more preferably the Group III to Group V base oils due to their exceptional volatility, stability, viscometric and cleanliness features. Minor quantities of Group I stock, such as the amount used to dilute additives for blending into formulated lube oil products, can be tolerated but should be kept to a minimum, i.e. amounts only associated with their use as diluent/carrier oil for additives used on an “as-received” basis. Even in regard to the Group II stocks, it is preferred that the Group II stock be in the higher quality range associated with that stock, i.e. a Group II stock having a viscosity index in the range 100<VI<120.

The base oil constitutes the major component of the engine oil lubricant composition of the present disclosure and typically is present in an amount ranging from about 50 to about 99 weight percent, preferably from about 70 to about 95 weight percent, and more preferably from about 85 to about 95 weight percent, based on the total weight of the composition. The base oil may be selected from any of the synthetic or natural oils typically used as crankcase lubricating oils for spark-ignited and compression-ignited engines. The base oil conveniently has a kinematic viscosity, according to ASTM standards, of about 2.5 cSt to about 12 cSt (or mm²/s) at 100° C. and preferably of about 2.5 cSt to about 9 cSt (or mm²/s) at 100° C. Mixtures of synthetic and natural base oils may be used if desired. Bi-modal mixtures of Group I, II, III, IV, and/or V base stocks may be used if desired.

### Additives

The formulated lubricating oil useful in the present disclosure may additionally contain one or more of commonly used lubricating oil performance additives including but not limited to detergents, antiwear additives, dispersants, viscosity modifiers, corrosion inhibitors, rust inhibitors, metal deactivators, extreme pressure additives, anti-seizure agents, wax modifiers, viscosity modifiers, fluid-loss additives, seal compatibility agents, lubricity agents, anti-staining agents, chromophoric agents, defoamants, demulsifiers, emulsifiers, densifiers, wetting agents, gelling agents, tackiness agents, colorants, and others. For a review of many commonly used additives, see Klamann in Lubricants and Related Products, Verlag Chemie, Deerfield Beach, Fla.; ISBN 0-89573-177-0. Reference is also made to “Lubricant Additives” by M. W. Ranney, published by Noyes Data Corporation of Parkridge, N.J. (1973); see also U.S. Pat. No. 7,704,930, the disclosure of which is incorporated herein in its entirety. These additives are commonly delivered with varying amounts of diluent oil, that may range from 5 weight percent to 50 weight percent.

The additives useful in this disclosure do not have to be soluble in the lubricating oils. Insoluble additives such as zinc stearate in oil can be dispersed in the lubricating oils of this disclosure.

The types and quantities of performance additives used in combination with the instant disclosure in lubricant compositions are not limited by the examples shown herein as illustrations.

### Detergents

Illustrative detergents useful in this disclosure include, for example, alkali metal detergents, alkaline earth metal detergents, or mixtures of one or more alkali metal detergents and one or more alkaline earth metal detergents. A typical detergent is an anionic material that contains a long chain hydrophobic portion of the molecule and a smaller anionic or oleophobic hydrophilic portion of the molecule. The anionic portion of the detergent is typically derived from an organic acid such as a sulfur acid, carboxylic acid (e.g., salicylic acid), phosphorous acid, phenol, or mixtures thereof. The counterion is typically an alkaline earth or alkali metal. The detergent can be overbased as described herein.

The detergent is preferably a metal salt of an organic or inorganic acid, a metal salt of a phenol, or mixtures thereof. The metal is preferably selected from an alkali metal, an alkaline earth metal, and mixtures thereof. The organic or inorganic acid is selected from an aliphatic organic or inorganic acid, a cycloaliphatic organic or inorganic acid, an aromatic organic or inorganic acid, and mixtures thereof.

The metal is preferably selected from an alkali metal, an alkaline earth metal, and mixtures thereof. More preferably, the metal is selected from calcium (Ca), magnesium (Mg), and mixtures thereof.

The organic acid or inorganic acid is preferably selected from a sulfur acid, a carboxylic acid, a phosphorus acid, and mixtures thereof.

Preferably, the metal salt of an organic or inorganic acid or the metal salt of a phenol comprises calcium phenate, calcium sulfonate, calcium salicylate, magnesium phenate, magnesium sulfonate, magnesium salicylate, an overbased detergent, and mixtures thereof.

Salts that contain a substantially stochiometric amount of the metal are described as neutral salts and have a total base number (TBN, as measured by ASTM D2896) of from 0 to 80. Many compositions are overbased, containing large amounts of a metal base that is achieved by reacting an excess of a metal compound (a metal hydroxide or oxide, for example) with an acidic gas (such as carbon dioxide). Useful detergents can be neutral, mildly overbased, or highly overbased. These detergents can be used in mixtures of neutral, overbased, highly overbased calcium salicylate, sulfonates, phenates and/or magnesium salicylate, sulfonates, phenates. The TBN ranges can vary from low, medium to high TBN products, including as low as 0 to as high as 600. Mixtures of low, medium, high TBN can be used, along with mixtures of calcium and magnesium metal based detergents, and including sulfonates, phenates, salicylates, and carboxylates. A detergent mixture with a metal ratio of 1, in conjunction of a detergent with a metal ratio of 2, and as high as a detergent with a metal ratio of 5, can be used. Borated detergents can also be used.

Alkaline earth phenates are another useful class of detergent. These detergents can be made by reacting alkaline earth metal hydroxide or oxide (CaO, Ca(OH)₂, BaO, Ba(OH)₂, MgO, Mg(OH)₂, for example) with an alkyl phenol or sulfurized alkylphenol. Useful alkyl groups include straight chain or branched C₁-C₃₀ alkyl groups, preferably, C₄-C₂₀ or mixtures thereof. Examples of suitable phenols include isobutylphenol, 2-ethylhexylphenol, nonylphenol, dodecyl phenol, and the like. It should be noted that starting alkylphenols may contain more than one alkyl substituent that are each independently straight chain or branched and can be used from 0.5 to 6 weight percent. When a non-sulfurized alkylphenol is used, the sulfurized product may be obtained by methods well known in the art. These methods include heating a mixture of alkylphenol and sulfurizing agent (including elemental sulfur, sulfur halides such as sulfur dichloride, and the like) and then reacting the sulfurized phenol with an alkaline earth metal base.

In accordance with this disclosure, metal salts of carboxylic acids are preferred detergents. These carboxylic acid detergents may be prepared by reacting a basic metal compound with at least one carboxylic acid and removing free water from the reaction product. These compounds may be overbased to produce the desired TBN level. Detergents made from salicylic acid are one preferred class of detergents derived from carboxylic acids. Useful salicylates include long chain alkyl salicylates. One useful family of compositions is of the formula

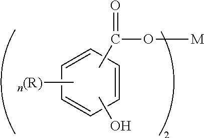

where R is an alkyl group having 1 to about 30 carbon atoms, n is an integer from 1 to 4, and M is an alkaline earth metal. Preferred R groups are alkyl chains of at least C₁₁, preferably C₁₃ or greater. R may be optionally substituted with substituents that do not interfere with the detergent's function. M is preferably, calcium, magnesium, or barium. More preferably, M is calcium.

Hydrocarbyl-substituted salicylic acids may be prepared from phenols by the Kolbe reaction (see U.S. Pat. No. 3,595,791). The metal salts of the hydrocarbyl-substituted salicylic acids may be prepared by double decomposition of a metal salt in a polar solvent such as water or alcohol.

Alkaline earth metal phosphates are also used as detergents and are known in the art.

Detergents may be simple detergents or what is known as hybrid or complex detergents. The latter detergents can provide the properties of two detergents without the need to blend separate materials. See U.S. Pat. No. 6,034,039.

Preferred detergents include calcium sulfonates, magnesium sulfonates, calcium salicylates, magnesium salicylates, calcium phenates, magnesium phenates, and other related components (including borated detergents), and mixtures thereof. Preferred mixtures of detergents include magnesium sulfonate and calcium salicylate, magnesium sulfonate and calcium sulfonate, magnesium sulfonate and calcium phenate, calcium phenate and calcium salicylate, calcium phenate and calcium sulfonate, calcium phenate and magnesium salicylate, calcium phenate and magnesium phenate. Overbased detergents are also preferred.

The detergent concentration in the lubricating oils of this disclosure can range from about 0.5 to about 6.0 weight percent, preferably about 0.6 to 5.0 weight percent, and more preferably from about 0.8 weight percent to about 4.0 weight percent, based on the total weight of the lubricating oil.

As used herein, the detergent concentrations are given on an “as delivered” basis. Typically, the active detergent is delivered with a process oil.

The “as delivered” detergent typically contains from about 20 weight percent to about 100 weight percent, or from about 40 weight percent to about 60 weight percent, of active detergent in the “as delivered” detergent product.

### Antiwear Additives

Illustrative antiwear additives useful in this disclosure include, for example, metal salts of a carboxylic acid. The metal is selected from a transition metal and mixtures thereof. The carboxylic acid is selected from an aliphatic carboxylic acid, a cycloaliphatic carboxylic acid, an aromatic carboxylic acid, and mixtures thereof.

The metal is preferably selected from a Group 10, 11 and 12 metal, and mixtures thereof. The carboxylic acid is preferably an aliphatic, saturated, unbranched carboxylic acid having from about 8 to about 26 carbon atoms, and mixtures thereof.

The metal is preferably selected from nickel (Ni), palladium (Pd), platinum (Pt), copper (Cu), silver (Ag), gold (Au), zinc (Zn), cadium (Cd), mercury (Hg), and mixtures thereof.

The carboxylic acid is preferably selected from caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), and mixtures thereof.

Preferably, the metal salt of a carboxylic acid comprises zinc stearate, silver stearate, palladium stearate, zinc palmitate, silver palmitate, palladium palmitate, and mixtures thereof.

The metal salt of a carboxylic acid is present in the engine oil formulations of this disclosure in an amount of from about 0.01 weight percent to about 5 weight percent, based on the total weight of the formulated oil.

Low phosphorus engine oil formulations are included in this disclosure. For such formulations, the phosphorus content is typically less than about 0.12 weight percent preferably less than about 0.10 weight percent and most preferably less than about 0.085 weight percent.

A metal alkylthiophosphate and more particularly a metal dialkyl dithio phosphate in which the metal constituent is zinc, or zinc dialkyl dithio phosphate (ZDDP) can be a useful component of the lubricating oils of this disclosure. ZDDP can be derived from primary alcohols, secondary alcohols or mixtures thereof. ZDDP compounds generally are of the formula

Zn[SP(S)(OR¹)(OR²)]₂

where R¹ and R² are C₁-C₁₈ alkyl groups, preferably C₂-C₁₂ alkyl groups. These alkyl groups may be straight chain or branched. Alcohols used in the ZDDP can be 2-propanol, butanol, secondary butanol, pentanols, hexanols such as 4-methyl-2-pentanol, n-hexanol, n-octanol, 2-ethyl hexanol, alkylated phenols, and the like. Mixtures of secondary alcohols or of primary and secondary alcohol can be preferred. Alkyl aryl groups may also be used.

Preferable zinc dithiophosphates which are commercially available include secondary zinc dithiophosphates such as those available from for example, The Lubrizol Corporation under the trade designations “LZ 677A”, “LZ 1095” and “LZ 1371”, from for example Chevron Oronite under the trade designation “OLOA 262” and from for example Afton Chemical under the trade designation “HITEC 7169”.

The ZDDP is typically used in amounts of from about 0.4 weight percent to about 1.2 weight percent, preferably from about 0.5 weight percent to about 1.0 weight percent, and more preferably from about 0.6 weight percent to about 0.8 weight percent, based on the total weight of the lubricating oil, although more or less can often be used advantageously. Preferably, the ZDDP is a secondary ZDDP and present in an amount of from about 0.6 to 1.0 weight percent of the total weight of the lubricating oil.

Low phosphorus engine oil formulations are included in this disclosure. For such formulations, the phosphorus content is typically less than about 0.12 weight percent preferably less than about 0.10 weight percent and most preferably less than about 0.085 weight percent.

### Dispersants

During engine operation, oil-insoluble oxidation byproducts are produced. Dispersants help keep these byproducts in solution, thus diminishing their deposition on metal surfaces. Dispersants used in the formulation of the lubricating oil may be ashless or ash-forming in nature. Preferably, the dispersant is ashless. So called ashless dispersants are organic materials that form substantially no ash upon combustion. For example, non-metal-containing or borated metal-free dispersants are considered ashless. In contrast, metal-containing detergents discussed above form ash upon combustion.

Suitable dispersants typically contain a polar group attached to a relatively high molecular weight hydrocarbon chain. The polar group typically contains at least one element of nitrogen, oxygen, or phosphorus. Typical hydrocarbon chains contain 50 to 400 carbon atoms.

A particularly useful class of dispersants are the (poly)alkenylsuccinic derivatives, typically produced by the reaction of a long chain hydrocarbyl substituted succinic compound, usually a hydrocarbyl substituted succinic anhydride, with a polyhydroxy or polyamino compound. The long chain hydrocarbyl group constituting the oleophilic portion of the molecule which confers solubility in the oil, is normally a polyisobutylene group. Many examples of this type of dispersant are well known commercially and in the literature. Exemplary U.S. patents describing such dispersants are U.S. Pat. Nos. 3,172,892; 3,2145,707; 3,219,666; 3,316,177; 3,341,542; 3,444,170; 3,454,607; 3,541,012; 3,630,904; 3,632,511; 3,787,374 and 4,234,435. Other types of dispersant are described in U.S. Pat. Nos. 3,036,003; 3,200,107; 3,254,025; 3,275,554; 3,438,757; 3,454,555; 3,565,804; 3,413,347; 3,697,574; 3,725,277; 3,725,480; 3,726,882; 4,454,059; 3,329,658; 3,449,250; 3,519,565; 3,666,730; 3,687,849; 3,702,300; 4,100,082; 5,705,458. A further description of dispersants may be found, for example, in European Patent Application No. 471 071, to which reference is made for this purpose.

Hydrocarbyl-substituted succinic acid and hydrocarbyl-substituted succinic anhydride derivatives are useful dispersants. In particular, succinimide, succinate esters, or succinate ester amides prepared by the reaction of a hydrocarbon-substituted succinic acid compound preferably having at least 50 carbon atoms in the hydrocarbon substituent, with at least one equivalent of an alkylene amine are particularly useful.

Succinimides are formed by the condensation reaction between hydrocarbyl substituted succinic anhydrides and amines. Molar ratios can vary depending on the polyamine. For example, the molar ratio of hydrocarbyl substituted succinic anhydride to TEPA can vary from about 1:1 to about 5:1. Representative examples are shown in U.S. Pat. Nos. 3,087,936; 3,172,892; 3,219,666; 3,272,746; 3,322,670; and 3,652,616, 3,948,800; and Canada Patent No. 1,094,044.

Succinate esters are formed by the condensation reaction between hydrocarbyl substituted succinic anhydrides and alcohols or polyols. Molar ratios can vary depending on the alcohol or polyol used. For example, the condensation product of a hydrocarbyl substituted succinic anhydride and pentaerythritol is a useful dispersant.

Succinate ester amides are formed by condensation reaction between hydrocarbyl substituted succinic anhydrides and alkanol amines. For example, suitable alkanol amines include ethoxylated polyalkylpolyamines, propoxylated polyalkylpolyamines and polyalkenylpolyamines such as polyethylene polyamines. One example is propoxylated hexamethylenediamine. Representative examples are shown in U.S. Pat. No. 4,426,305.

The molecular weight of the hydrocarbyl substituted succinic anhydrides used in the preceding paragraphs will typically range between 800 and 2,500 or more. The above products can be post-reacted with various reagents such as sulfur, oxygen, formaldehyde, carboxylic acids such as oleic acid. The above products can also be post reacted with boron compounds such as boric acid, borate esters or highly borated dispersants, to form borated dispersants generally having from about 0.1 to about 5 moles of boron per mole of dispersant reaction product.

Mannich base dispersants are made from the reaction of alkylphenols, formaldehyde, and amines. See U.S. Pat. No. 4,767,551, which is incorporated herein by reference. Process aids and catalysts, such as oleic acid and sulfonic acids, can also be part of the reaction mixture. Molecular weights of the alkylphenols range from 800 to 2,500. Representative examples are shown in U.S. Pat. Nos. 3,697,574; 3,703,536; 3,704,308; 3,751,365; 3,756,953; 3,798,165; and 3,803,039.

Typical high molecular weight aliphatic acid modified Mannich condensation products useful in this disclosure can be prepared from high molecular weight alkyl-substituted hydroxyaromatics or HNR₂ group-containing reactants.

Hydrocarbyl substituted amine ashless dispersant additives are well known to one skilled in the art; see, for example, U.S. Pat. Nos. 3,275,554; 3,438,757; 3,565,804; 3,755,433, 3,822,209, and 5,084,197.

Preferred dispersants include borated and non-borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having a Mn of from about 500 to about 5000, or from about 1000 to about 3000, or about 1000 to about 2000, or a mixture of such hydrocarbylene groups, often with high terminal vinylic groups. Other preferred dispersants include succinic acid-esters and amides, alkylphenol-polyamine-coupled Mannich adducts, their capped derivatives, and other related components.

Polymethacrylate or polyacrylate derivatives are another class of dispersants. These dispersants are typically prepared by reacting a nitrogen containing monomer and a methacrylic or acrylic acid esters containing 5-25 carbon atoms in the ester group. Representative examples are shown in U.S. Pat. Nos. 2,100,993, and 6,323,164. Polymethacrylate and polyacrylate dispersants are normally used as multifunctional viscosity modifiers. The lower molecular weight versions can be used as lubricant dispersants or fuel detergents.

Illustrative preferred dispersants useful in this disclosure include those derived from polyalkenyl-substituted mono- or dicarboxylic acid, anhydride or ester, which dispersant has a polyalkenyl moiety with a number average molecular weight of at least 900 and from greater than 1.3 to 1.7, preferably from greater than 1.3 to 1.6, most preferably from greater than 1.3 to 1.5, functional groups (mono- or dicarboxylic acid producing moieties) per polyalkenyl moiety (a medium functionality dispersant). Functionality (F) can be determined according to the following formula:

F=(SAP×Mₙ)/((112,200×A.I.)−(SAP×98))

wherein SAP is the saponification number (i.e., the number of milligrams of KOH consumed in the complete neutralization of the acid groups in one gram of the succinic-containing reaction product, as determined according to ASTM D94); Mₙ is the number average molecular weight of the starting olefin polymer; and A.I. is the percent active ingredient of the succinic-containing reaction product (the remainder being unreacted olefin polymer, succinic anhydride and diluent).

The polyalkenyl moiety of the dispersant may have a number average molecular weight of at least 900, suitably at least 1500, preferably between 1800 and 3000, such as between 2000 and 2800, more preferably from about 2100 to 2500, and most preferably from about 2200 to about 2400. The molecular weight of a dispersant is generally expressed in terms of the molecular weight of the polyalkenyl moiety. This is because the precise molecular weight range of the dispersant depends on numerous parameters including the type of polymer used to derive the dispersant, the number of functional groups, and the type of nucleophilic group employed.

Polymer molecular weight, specifically Mₙ, can be determined by various known techniques. One convenient method is gel permeation chromatography (GPC), which additionally provides molecular weight distribution information (see W. W. Yau, J. J. Kirkland and D. D. Bly, “Modern Size Exclusion Liquid Chromatography”, John Wiley and Sons, New York, 1979). Another useful method for determining molecular weight, particularly for lower molecular weight polymers, is vapor pressure osmometry (e.g., ASTM D3592).

The polyalkenyl moiety in a dispersant preferably has a narrow molecular weight distribution (MWD), also referred to as polydispersity, as determined by the ratio of weight average molecular weight (M_{w}) to number average molecular weight (Mₙ). Polymers having a M_{w}/Mₙ of less than 2.2, preferably less than 2.0, are most desirable. Suitable polymers have a polydispersity of from about 1.5 to 2.1, preferably from about 1.6 to about 1.8.

Suitable polyalkenes employed in the formation of the dispersants include homopolymers, interpolymers or lower molecular weight hydrocarbons. One family of such polymers comprise polymers of ethylene and/or at least one C₃ to C₂ alpha-olefin having the formula H₂C═CHR¹ wherein R¹ is a straight or branched chain alkyl radical comprising 1 to 26 carbon atoms and wherein the polymer contains carbon-to-carbon unsaturation, and a high degree of terminal ethenylidene unsaturation. Preferably, such polymers comprise interpolymers of ethylene and at least one alpha-olefin of the above formula, wherein R¹ is alkyl of from 1 to 18 carbon atoms, and more preferably is alkyl of from 1 to 8 carbon atoms, and more preferably still of from 1 to 2 carbon atoms.

Another useful class of polymers is polymers prepared by cationic polymerization of monomers such as isobutene and styrene. Common polymers from this class include polyisobutenes obtained by polymerization of a C₄ refinery stream having a butene content of 35 to 75% by wt., and an isobutene content of 30 to 60% by wt. A preferred source of monomer for making poly-n-butenes is petroleum feedstreams such as Raffinate II. These feedstocks are disclosed in the art such as in U.S. Pat. No. 4,952,739. A preferred embodiment utilizes polyisobutylene prepared from a pure isobutylene stream or a Raffinate I stream to prepare reactive isobutylene polymers with terminal vinylidene olefins. Polyisobutene polymers that may be employed are generally based on a polymer chain of from 1500 to 3000.

The dispersant(s) are preferably non-polymeric (e.g., mono- or bis-succinimides). Such dispersants can be prepared by conventional processes such as disclosed in U.S. Patent Application Publication No. 2008/0020950, the disclosure of which is incorporated herein by reference.

The dispersant(s) can be borated by conventional means, as generally disclosed in U.S. Pat. Nos. 3,087,936, 3,254,025 and 5,430,105.

Such dispersants may be used in an amount of about 0.01 to 20 weight percent or 0.01 to 10 weight percent, preferably about 0.5 to 8 weight percent, or more preferably 0.5 to 4 weight percent. Or such dispersants may be used in an amount of about 2 to 12 weight percent, preferably about 4 to 10 weight percent, or more preferably 6 to 9 weight percent. On an active ingredient basis, such additives may be used in an amount of about 0.06 to 14 weight percent, preferably about 0.3 to 6 weight percent. The hydrocarbon portion of the dispersant atoms can range from C₆₀ to C₁₀₀₀, or from C₇₀ to C₃₀₀, or from C₇₀ to C₂₀₀. These dispersants may contain both neutral and basic nitrogen, and mixtures of both. Dispersants can be end-capped by borates and/or cyclic carbonates.

As used herein, the dispersant concentrations are given on an “as delivered” basis. Typically, the active dispersant is delivered with a process oil. The “as delivered” dispersant typically contains from about 20 weight percent to about 80 weight percent, or from about 40 weight percent to about 60 weight percent, of active dispersant in the “as delivered” dispersant product.

### Viscosity Modifiers

Viscosity modifiers (also known as viscosity index improvers (VI improvers), and viscosity improvers) can be included in the lubricant compositions of this disclosure.

Viscosity modifiers provide lubricants with high and low temperature operability. These additives impart shear stability at elevated temperatures and acceptable viscosity at low temperatures.

Suitable viscosity modifiers include high molecular weight hydrocarbons, polyesters and viscosity modifier dispersants that function as both a viscosity modifier and a dispersant. Typical molecular weights of these polymers are between about 10,000 to 1,500,000, more typically about 20,000 to 1,200,000, and even more typically between about 50,000 and 1,000,000.

Examples of suitable viscosity modifiers are linear or star-shaped polymers and copolymers of methacrylate, butadiene, olefins, or alkylated styrenes. Polyisobutylene is a commonly used viscosity modifier. Another suitable viscosity modifier is polymethacrylate (copolymers of various chain length alkyl methacrylates, for example), some formulations of which also serve as pour point depressants. Other suitable viscosity modifiers include copolymers of ethylene and propylene, hydrogenated block copolymers of styrene and isoprene, and polyacrylates (copolymers of various chain length acrylates, for example). Specific examples include styrene-isoprene or styrene-butadiene based polymers of 50,000 to 200,000 molecular weight.

Olefin copolymers are commercially available from Chevron Oronite Company LLC under the trade designation “PARATONE®” (such as “PARATONE® 8921” and “PARATONE® 8941”); from Afton Chemical Corporation under the trade designation “HiTEC®” (such as “HiTEC® 5850B”; and from The Lubrizol Corporation under the trade designation “Lubrizol® 7067C”. Hydrogenated polyisoprene star polymers are commercially available from Infineum International Limited, e.g., under the trade designation “SV200” and “SV600”. Hydrogenated diene-styrene block copolymers are commercially available from Infineum International Limited, e.g., under the trade designation “SV 50”.

The polymethacrylate or polyacrylate polymers can be linear polymers which are available from Evnoik Industries under the trade designation “Viscoplex®” (e.g., Viscoplex 6-954) or star polymers which are available from Lubrizol Corporation under the trade designation Asteric™ (e.g., Lubrizol 87708 and Lubrizol 87725).

Illustrative vinyl aromatic-containing polymers useful in this disclosure may be derived predominantly from vinyl aromatic hydrocarbon monomer. Illustrative vinyl aromatic-containing copolymers useful in this disclosure may be represented by the following general formula:

A-B

wherein A is a polymeric block derived predominantly from vinyl aromatic hydrocarbon monomer, and B is a polymeric block derived predominantly from conjugated diene monomer.

In an embodiment of this disclosure, the viscosity modifiers may be used in an amount of less than about 2.0 weight percent, preferably less than about 1.0 weight percent, and more preferably less than about 0.5 weight percent, based on the total weight of the formulated oil or lubricating engine oil. Viscosity modifiers are typically added as concentrates, in large amounts of diluent oil.

As used herein, the viscosity modifier concentrations are given on an “as delivered” basis. Typically, the active polymer is delivered with a diluent oil. The “as delivered” viscosity modifier typically contains from 20 weight percent to 75 weight percent of an active polymer for polymethacrylate or polyacrylate polymers, or from 8 weight percent to 20 weight percent of an active polymer for olefin copolymers, hydrogenated polyisoprene star polymers, or hydrogenated diene-styrene block copolymers, in the “as delivered” polymer concentrate.

### Antioxidants

Antioxidants retard the oxidative degradation of base oils during service. Such degradation may result in deposits on metal surfaces, the presence of sludge, or a viscosity increase in the lubricant. One skilled in the art knows a wide variety of oxidation inhibitors that are useful in lubricating oil compositions. See, Klamann in Lubricants and Related Products, op cite, and U.S. Pat. Nos. 4,798,684 and 5,084,197, for example.

Useful antioxidants include hindered phenols. These phenolic antioxidants may be ashless (metal-free) phenolic compounds or neutral or basic metal salts of certain phenolic compounds. Typical phenolic antioxidant compounds are the hindered phenolics which are the ones which contain a sterically hindered hydroxyl group, and these include those derivatives of dihydroxy aryl compounds in which the hydroxyl groups are in the o- or p-position to each other. Typical phenolic antioxidants include the hindered phenols substituted with C₆+ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; and 2-methyl-6-t-butyl-4-dodecyl phenol. Other useful hindered mono-phenolic antioxidants may include for example hindered 2,6-di-alkyl-phenolic proprionic ester derivatives. Bis-phenolic antioxidants may also be advantageously used in combination with the instant disclosure. Examples of ortho-coupled phenols include: 2,2′-bis(4-heptyl-6-t-butyl-phenol); 2,2′-bis(4-octyl-6-t-butyl-phenol); and 2,2′-bis(4-dodecyl-6-t-butyl-phenol). Para-coupled bisphenols include for example 4,4′-bis(2,6-di-t-butyl phenol) and 4,4′-methylene-bis(2,6-di-t-butyl phenol).

Effective amounts of one or more catalytic antioxidants may also be used. The catalytic antioxidants comprise an effective amount of a) one or more oil soluble polymetal organic compounds; and, effective amounts of b) one or more substituted N,N′-diaryl-o-phenylenediamine compounds or c) one or more hindered phenol compounds; or a combination of both b) and c). Catalytic antioxidants are more fully described in U.S. Pat. No. 8,048,833, herein incorporated by reference in its entirety.

Non-phenolic oxidation inhibitors which may be used include aromatic amine antioxidants and these may be used either as such or in combination with phenolics. Typical examples of non-phenolic antioxidants include: alkylated and non-alkylated aromatic amines such as aromatic monoamines of the formula R⁸R⁹R¹⁰N where R⁸ is an aliphatic, aromatic or substituted aromatic group, R⁹ is an aromatic or a substituted aromatic group, and R¹⁰ is H, alkyl, aryl or R¹¹S(O)_{X}R¹² where R¹¹ is an alkylene, alkenylene, or aralkylene group, R¹² is a higher alkyl group, or an alkenyl, aryl, or alkaryl group, and x is 0, 1 or 2. The aliphatic group R⁸ may contain from 1 to about 20 carbon atoms, and preferably contains from about 6 to 12 carbon atoms. The aliphatic group is a saturated aliphatic group. Preferably, both R⁸ and R⁹ are aromatic or substituted aromatic groups, and the aromatic group may be a fused ring aromatic group such as naphthyl. Aromatic groups R⁸ and R⁹ may be joined together with other groups such as S.

Typical aromatic amines antioxidants have alkyl substituent groups of at least about 6 carbon atoms. Examples of aliphatic groups include hexyl, heptyl, octyl, nonyl, and decyl. Generally, the aliphatic groups will not contain more than about 14 carbon atoms. The general types of amine antioxidants useful in the present compositions include diphenylamines, phenyl naphthylamines, phenothiazines, imidodibenzyls and diphenyl phenylene diamines. Mixtures of two or more aromatic amines are also useful. Polymeric amine antioxidants can also be used. Particular examples of aromatic amine antioxidants useful in the present disclosure include: p,p′-dioctyldiphenylamine; t-octylphenyl-alpha-naphthylamine; phenyl-alphanaphthylamine; and p-octylphenyl-alpha-naphthylamine.

Sulfurized alkyl phenols and alkali or alkaline earth metal salts thereof also are useful antioxidants.

Preferred antioxidants include hindered phenols, arylamines. These antioxidants may be used individually by type or in combination with one another. Such additives may be used in an amount of about 0.01 to 5 weight percent, preferably about 0.01 to 1.5 weight percent, more preferably zero to less than 1.5 weight percent, more preferably zero to less than 1 weight percent.

### Pour Point Depressants (PPDs)

Conventional pour point depressants (also known as lube oil flow improvers) may be added to the compositions of the present disclosure if desired. These pour point depressant may be added to lubricating compositions of the present disclosure to lower the minimum temperature at which the fluid will flow or can be poured. Examples of suitable pour point depressants include polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. U.S. Pat. Nos. 1,815,022; 2,015,748; 2,191,498; 2,387,501; 2,655, 479; 2,666,746; 2,721,877; 2,721,878; and 3,250,715 describe useful pour point depressants and/or the preparation thereof. Such additives may be used in an amount of about 0.01 to 5 weight percent, preferably about 0.01 to 1.5 weight percent.

### Seal Compatibility Agents

Seal compatibility agents help to swell elastomeric seals by causing a chemical reaction in the fluid or physical change in the elastomer. Suitable seal compatibility agents for lubricating oils include organic phosphates, aromatic esters, aromatic hydrocarbons, esters (butylbenzyl phthalate, for example), and polybutenyl succinic anhydride. Such additives may be used in an amount of about 0.01 to 3 weight percent, preferably about 0.01 to 2 weight percent.

### Antifoam Agents

Anti-foam agents may advantageously be added to lubricant compositions. These agents retard the formation of stable foams. Silicones and organic polymers are typical anti-foam agents. For example, polysiloxanes, such as silicon oil or polydimethyl siloxane, provide antifoam properties. Anti-foam agents are commercially available and may be used in conventional minor amounts along with other additives such as demulsifiers; usually the amount of these additives combined is less than 1 weight percent and often less than 0.1 weight percent.

### Inhibitors and Antirust Additives

Antirust additives (or corrosion inhibitors) are additives that protect lubricated metal surfaces against chemical attack by water or other contaminants. A wide variety of these are commercially available.

One type of antirust additive is a polar compound that wets the metal surface preferentially, protecting it with a film of oil. Another type of antirust additive absorbs water by incorporating it in a water-in-oil emulsion so that only the oil touches the metal surface. Yet another type of antirust additive chemically adheres to the metal to produce a non-reactive surface. Examples of suitable additives include zinc dithiophosphates, metal phenolates, basic metal sulfonates, fatty acids and amines. Such additives may be used in an amount of about 0.01 to 5 weight percent, preferably about 0.01 to 1.5 weight percent.

### Friction Modifiers

A friction modifier is any material or materials that can alter the coefficient of friction of a surface lubricated by any lubricant or fluid containing such material(s). Friction modifiers, also known as friction reducers, or lubricity agents or oiliness agents, and other such agents that change the ability of base oils, formulated lubricant compositions, or functional fluids, to modify the coefficient of friction of a lubricated surface may be effectively used in combination with the base oils or lubricant compositions of the present disclosure if desired. Friction modifiers that lower the coefficient of friction are particularly advantageous in combination with the base oils and lube compositions of this disclosure.

Illustrative friction modifiers may include, for example, organometallic compounds or materials, or mixtures thereof. Illustrative organometallic friction modifiers useful in the lubricating engine oil formulations of this disclosure include, for example, molybdenum amine, molybdenum diamine, an organotungstenate, a molybdenum dithiocarbamate, molybdenum dithiophosphates, molybdenum amine complexes, molybdenum carboxylates, and the like, and mixtures thereof. Similar tungsten based compounds may be preferable.

Other illustrative friction modifiers useful in the lubricating engine oil formulations of this disclosure include, for example, alkoxylated fatty acid esters, alkanolamides, polyol fatty acid esters, borated glycerol fatty acid esters, fatty alcohol ethers, and mixtures thereof.

Illustrative alkoxylated fatty acid esters include, for example, polyoxyethylene stearate, fatty acid polyglycol ester, and the like. These can include polyoxypropylene stearate, polyoxybutylene stearate, polyoxyethylene isosterate, polyoxypropylene isostearate, polyoxyethylene palmitate, and the like.

Illustrative alkanolamides include, for example, lauric acid diethylalkanolamide, palmic acid diethylalkanolamide, and the like. These can include oleic acid diethyalkanolamide, stearic acid diethylalkanolamide, oleic acid diethylalkanolamide, polyethoxylated hydrocarbylamides, polypropoxylated hydrocarbylamides, and the like.

Illustrative polyol fatty acid esters include, for example, glycerol mono-oleate, saturated mono-, di-, and tri-glyceride esters, glycerol mono-stearate, and the like. These can include polyol esters, hydroxyl-containing polyol esters, and the like.

Illustrative borated glycerol fatty acid esters include, for example, borated glycerol mono-oleate, borated saturated mono-, di-, and tri-glyceride esters, borated glycerol mono-sterate, and the like. In addition to glycerol polyols, these can include trimethylolpropane, pentaerythritol, sorbitan, and the like. These esters can be polyol monocarboxylate esters, polyol dicarboxylate esters, and on occasion polyoltricarboxylate esters. Preferred can be the glycerol mono-oleates, glycerol dioleates, glycerol trioleates, glycerol monostearates, glycerol distearates, and glycerol tristearates and the corresponding glycerol monopalmitates, glycerol dipalmitates, and glycerol tripalmitates, and the respective isostearates, linoleates, and the like. On occasion the glycerol esters can be preferred as well as mixtures containing any of these. Ethoxylated, propoxylated, butoxylated fatty acid esters of polyols, especially using glycerol as underlying polyol can be preferred.

Illustrative fatty alcohol ethers include, for example, stearyl ether, myristyl ether, and the like. Alcohols, including those that have carbon numbers from C₃ to C₅₀, can be ethoxylated, propoxylated, or butoxylated to form the corresponding fatty alkyl ethers. The underlying alcohol portion can preferably be stearyl, myristyl, C₁₁-C₁₃ hydrocarbon, oleyl, isosteryl, and the like.

The lubricating oils of this disclosure exhibit desired properties, e.g., wear control, in the presence or absence of a friction modifier.

Useful concentrations of friction modifiers may range from 0.01 weight percent to 5 weight percent, or about 0.1 weight percent to about 2.5 weight percent, or about 0.1 weight percent to about 1.5 weight percent, or about 0.1 weight percent to about 1 weight percent. Concentrations of molybdenum-containing materials are often described in terms of Mo metal concentration. Advantageous concentrations of Mo may range from 25 ppm to 700 ppm or more, and often with a preferred range of 50-200 ppm. Friction modifiers of all types may be used alone or in mixtures with the materials of this disclosure. Often mixtures of two or more friction modifiers, or mixtures of friction modifier(s) with alternate surface active material(s), are also desirable.

When lubricating oil compositions contain one or more of the additives discussed above, the additive(s) are blended into the composition in an amount sufficient for it to perform its intended function. Typical amounts of such additives useful in the present disclosure are shown in Table 1 below.

It is noted that many of the additives are shipped from the additive manufacturer as a concentrate, containing one or more additives together, with a certain amount of base oil diluents. Accordingly, the weight amounts in the table below, as well as other amounts mentioned herein, are directed to the amount of active ingredient (that is the non-diluent portion of the ingredient). The weight percent (wt %) indicated below is based on the total weight of the lubricating oil composition.

TABLE 1
Typical Amounts of Other Lubricating Oil Components

| | Approximate | Approximate |
|---|---|---|
| | wt % | wt % |
| Compound | (Useful) | (Preferred) |
| Dispersant | 0.1-20 | 0.1-8 |
| Detergent | 0.1-20 | 0.1-8 |
| Friction Modifier | 0.01-5 | 0.01-1.5 |
| Antioxidant | 0.1-5 | 0.1-1.5 |
| Pour Point Depressant | 0.0-5 | 0.01-1.5 |
| (PPD) | | |
| Anti-foam Agent | 0.001-3 | 0.001-0.15 |
| Viscosity Modifier (solid | 0.1-2 | 0.1-1 |
| polymer basis) | | |
| Antiwear | 0.2-3 | 0.5-1 |
| Inhibitor and Antirust | 0.01-5 | 0.01-1.5 |

The foregoing additives are all commercially available materials. These additives may be added independently but are usually precombined in packages which can be obtained from suppliers of lubricant oil additives. Additive packages with a variety of ingredients, proportions and characteristics are available and selection of the appropriate package will take the requisite use of the ultimate composition into account.

The following non-limiting examples are provided to illustrate the disclosure.

### Examples

An orthogonal set of taggants (i.e., sensor additives) which have been chosen as inert, but particularly responsive to certain stimuli are developed. These molecules (e.g. A: acid conditions, B: basic conditions, C: thermal excursions above 220° C., etc.) form the basis set for the analytical technique and aid in equipment condition diagnosis. For example, a used lubricant contains the entire sensor basis set at manufacture, but after one year of use in an industrial applications, the lubricant contains only molecules B and C at detectable levels. The system can then be diagnosed as suffering from an overly acidic environment and therefore an overbased lubricant should be considered for the application.

The bench test for correlation is a reactor vessel held at 120° C. for 72 hours (Oxidation Screener A). The sensor molecule used was a cyclic amide that is moderately stable to oxidation and detectable in lubricant by immunoassay at very low levels (<1 ppm). However, the sensor molecule decomposes slowly at elevated temperatures. Without knowing the exact kinetic model of the degradation, the initial concentration of the molecule can then be tuned such that the concentration of molecule of After Screener A is at the detection limit of the immunoassay. Once in service, a lubricant that contains a calibrated spike of the cyclic amide tag molecule can be used to interrogate whether the lubricant has experienced thermal/oxidative stresses equivalent to Screener A through the presence or absence of the molecule.

Evidence that the concentration of the molecular sensor can be tuned to enable either detection or absence after a bench screener (72 hours, 120° C.) is shown below.

| | As Received | After 72 Hrs, 120° C. |
|---|---|---|
| 10 ppm | Detect | Detect |
| molecular sensor | | |
| 0.5 ppm | Detect | No Detect |
| molecular sensor | | |

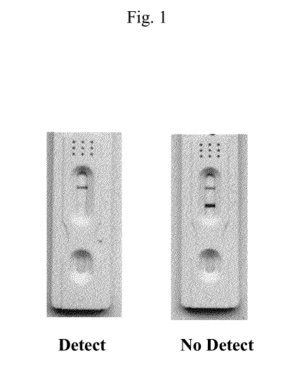

FIG. 1 shows examples of “detect” and “no detect” from immunoassay interrogation of a lubricating oil using a taggant in accordance with this disclosure.

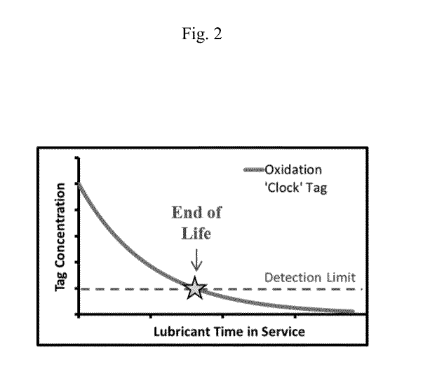

FIG. 2 graphically shows the use of taggants to detect oxidation in accordance with this disclosure.

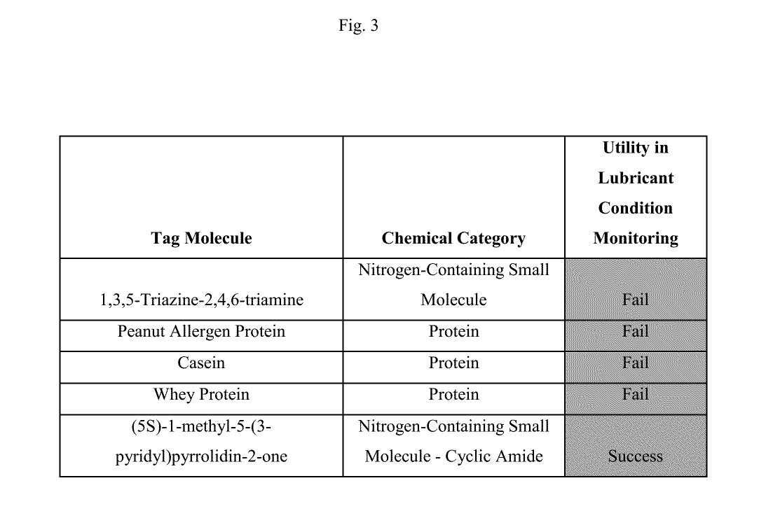

FIG. 3 shows examples of tag molecules that can be used in lubricants and their utility in determining the condition and/or identity of lubricating oils in accordance with this disclosure.

### Procedure for Immunoassay for Condition Monitoring

A typical procedure for immunoassay for condition monitoring involves four steps. The first step involves identifying an immunoassay taggant. The taggant should be compatible with the lubricant matrix, stable in the lubricant matrix, extractable into aqueous media, does not impact lubricant performance, and decomposes at similar time scales to the lubricant componentry.

The second step involves choosing oxidation screener conditions that correlate with end of the lubricant life (e.g., 144 hours in IIIE screener at 130° C. with metal catalyst).

The third step involves determining minimum detectable concentration of taggant (step 1) producing “no detect” after exposure to oxidation screener (step 2).

The fourth step involves using correlation developed (step 3) to easily and rapidly monitor the lubricant oxidation condition.

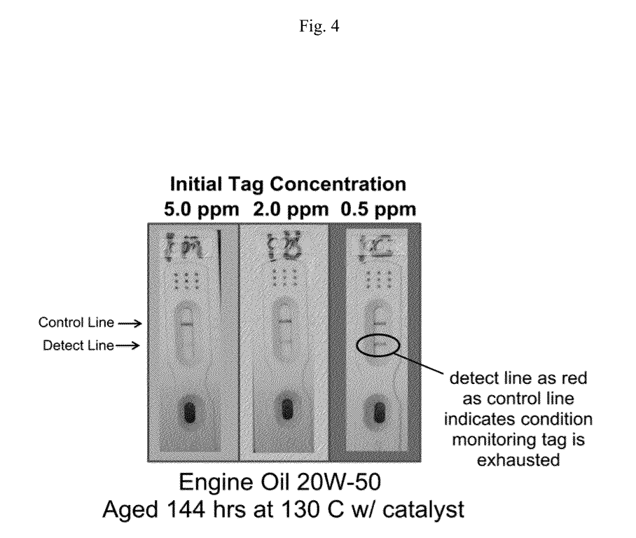

FIG. 4 shows examples of detection from immunoassay interrogation of a lubricating oil using a taggant (i.e., screening for initial concentration) in accordance with step 3 above.

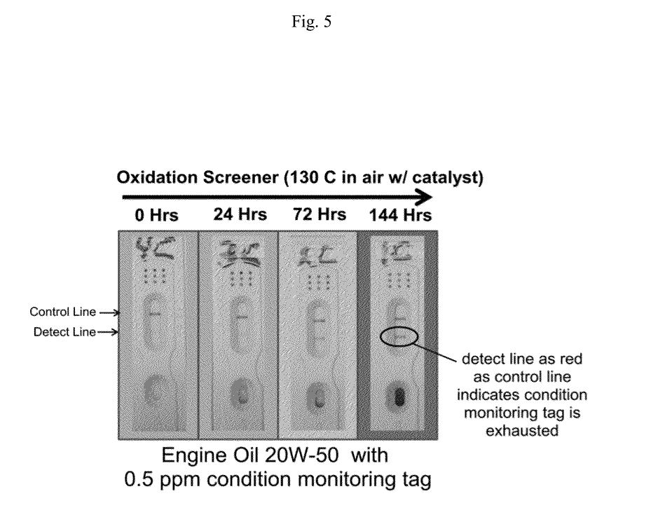

FIG. 5 shows examples of detection from immunoassay interrogation of a lubricating oil using a taggant (i.e., use of immunoassay to monitor lubricant condition) in accordance with step 4 above.

### PCT and EP Clauses:

1. A method for determining the condition of a product, said method comprising:

adding to the product a taggant; wherein the taggant exhibits degradation in response to one or more stimuli;

carrying out an immunoassay specific for the taggant to determine degradation of the taggant; and

determining the condition of the product based on the degradation of the taggant.

2. A method for monitoring degradation of a product, said method comprising:

adding to the product a taggant; wherein the taggant exhibits degradation in response to one or more stimuli; and

carrying out an immunoassay specific for the taggant to determine degradation of the taggant.

3. The method of clause 2 further comprising

identifying the condition of the product based on the degradation of the taggant.

4. The method of clauses 1-3 wherein the one or more stimuli comprise acidic conditions, basic or caustic conditions, thermal excursions, reductive/oxidative conditions, photochemical conditions, and contamination from another source.

5. The method of clauses 1-4 wherein the immunoassay is carried out using a test strip that is specific for the taggant.

6. The method of clauses 1-5 wherein the test strip is a lateral flow immunoassay.

7. The method of clauses 1-6 wherein the taggant comprises one or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

8. The method of clauses 1-7 wherein the taggant comprises a taggant array, and wherein the taggant array comprises two or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

9. The method of clauses 1-8 wherein the taggant comprises one or more aliphatic amide compounds or one or more cyclic amide compounds.

10. The method of clauses 1-9 wherein the taggant array comprises (i) two or more aliphatic amide compounds, (ii) two or more cyclic amide compounds, or (iii) a mixture of at least one aliphatic amide compound and at least one cyclic amide compound.

11. The method of clauses 9 and 10 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

12. The method of clauses 9 and 10 wherein the cyclic amide compounds are selected from (5 S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7, 8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

13. The method of clauses 1-12 wherein the product is selected from the group consisting of lubricating oils, automatic transmission fluids, engine oils, traction drive transmission fluids, manual transmission fluids, power steering fluids, antifreeze fluids, greases, crankcase lubricants, mineral oils, oils with Group 1, 2, 3 or 4 base oils, differential lubricants, turbine lubricants, gear lubricants, gear box lubricants, axle lubricants, brake fluids, farm tractor fluids, transformer fluids, compressor fluids, cooling system fluids, metal working fluids, hydraulic fluids, industrial fluids, fuels, continuously variable transmission fluid, infinitely variable transmission fluids, and mixtures thereof.

14. A method comprising:

associating a taggant with a product to produce a signature product; wherein the taggant exhibits degradation in response to one or more stimuli;

identifying the taggant in the signature product by an immunoassay specific for the taggant;

mapping the taggant of the signature product to a product code or a batch code of the signature product;

obtaining a test product to determine the condition and/or identity of the test product;

identifying the presence or absence of a taggant in the test product by an immunoassay specific for the taggant; and

comparing results of the immunoassay carried out on the test product with results of the immunoassay carried out on the signature product to determine the condition and/or identity of the test product.

15. A lubricating engine oil having a composition comprising a lubricating oil base stock as a major component; and a taggant, as a minor component; wherein the taggant exhibits degradation in response to one or more stimuli; and wherein the taggant is present in an amount sufficient for an immunoassay to be carried out specific for the taggant to determine degradation of the taggant.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in this art in light of the above detailed description. All such obvious variations are within the full intended scope of the appended claims.

What is claimed is:

1. A method for determining the condition of a product, said method comprising:
adding to the product a taggant; wherein the taggant exhibits degradation in response to one or more stimuli;
carrying out an immunoassay specific for the taggant to determine degradation of the taggant; and
determining the condition of the product based on the degradation of the taggant.

2. The method of claim 1 wherein the one or more stimuli comprise acidic conditions, basic or caustic conditions, thermal excursions, reductive/oxidative conditions, photochemical conditions, and contamination from another source.

3. The method of claim 1 wherein the immunoassay is carried out using a test strip that is specific for the taggant.

4. The method of claim 3 wherein the test strip is a lateral flow immunoassay.

5. The method of claim 1 wherein the taggant comprises one or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

6. The method of claim 1 wherein the taggant comprises a taggant array, and wherein the taggant array comprises two or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

7. The method of claim 5 wherein the taggant comprises one or more aliphatic amide compounds or one or more cyclic amide compounds.

8. The method of claim 7 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

9. The method of claim 7 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

10. The method of claim 6 wherein the taggant array comprises (i) two or more aliphatic amide compounds, (ii) two or more cyclic amide compounds, or (iii) a mixture of at least one aliphatic amide compound and at least one cyclic amide compound.

11. The method of claim 10 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

12. The method of claim 10 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

13. The method of claim 1 wherein the taggant is present in an amount of from 0.05 ppm to 20 ppm.

14. The method of claim 1 wherein the product is selected from the group consisting of lubricating oils, automatic transmission fluids, engine oils, traction drive transmission fluids, manual transmission fluids, power steering fluids, antifreeze fluids, greases, crankcase lubricants, mineral oils, oils with Group 1, 2, 3 or 4 base oils, differential lubricants, turbine lubricants, gear lubricants, gear box lubricants, axle lubricants, brake fluids, farm tractor fluids, transformer fluids, compressor fluids, cooling system fluids, metal working fluids, hydraulic fluids, industrial fluids, fuels, continuously variable transmission fluid, infinitely variable transmission fluids, and mixtures thereof.

15. The method of claim 1 wherein the product is a lubricating oil.

16. A method for monitoring degradation of a product, said method comprising:
adding to the product a taggant; wherein the taggant exhibits degradation in response to one or more stimuli; and
carrying out an immunoassay specific for the taggant to determine degradation of the taggant.

17. The method of claim 16 further comprising:
identifying the condition of the product based on the degradation of the taggant.

18. The method of claim 16 wherein the one or more stimuli comprise acidic conditions, basic or caustic conditions, thermal excursions, reductive/oxidative conditions, photochemical conditions, and contamination from another source.

19. The method of claim 16 wherein the immunoassay is carried out using a test strip that is specific for the taggant.

20. The method of claim 19 wherein the test strip is a lateral flow immunoassay.

21. The method of claim 16 wherein the taggant comprises one or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

22. The method of claim 16 wherein the taggant comprises a taggant array, and wherein the taggant array comprises two or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

23. The method of claim 21 wherein the taggant comprises one or more aliphatic amide compounds or one or more cyclic amide compounds.

24. The method of claim 23 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

25. The method of claim 23 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3 S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

26. The method of claim 22 wherein the taggant array comprises (i) two or more aliphatic amide compounds, (ii) two or more cyclic amide compounds, or (iii) a mixture of at least one aliphatic amide compound and at least one cyclic amide compound.

27. The method of claim 26 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

28. The method of claim 26 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

29. The method of claim 16 wherein the taggant is present in an amount of from 0.05 ppm to 20 ppm.

30. The method of claim 16 wherein the product is selected from the group consisting of lubricating oils, automatic transmission fluids, engine oils, traction drive transmission fluids, manual transmission fluids, power steering fluids, antifreeze fluids, greases, crankcase lubricants, mineral oils, oils with Group 1, 2, 3 or 4 base oils, differential lubricants, turbine lubricants, gear lubricants, gear box lubricants, axle lubricants, brake fluids, farm tractor fluids, transformer fluids, compressor fluids, cooling system fluids, metal working fluids, hydraulic fluids, industrial fluids, fuels, continuously variable transmission fluid, infinitely variable transmission fluids, and mixtures thereof.

31. The method of claim 16 wherein the product is a lubricating oil.

32. A method comprising:
associating a taggant with a product to produce a signature product; wherein the taggant exhibits degradation in response to one or more stimuli;
identifying the taggant in the signature product by an immunoassay specific for the taggant;
mapping the taggant of the signature product to a product code or a batch code of the signature product;
obtaining a test product to determine the condition and/or identity of the test product;
identifying the presence or absence of a taggant in the test product by an immunoassay specific for the taggant; and
comparing results of the immunoassay carried out on the test product with results of the immunoassay carried out on the signature product to determine the condition and/or identity of the test product.

33. The method of claim 32 wherein the one or more stimuli comprise acidic conditions, basic or caustic conditions, thermal excursions, reductive/oxidative conditions, photochemical conditions, and contamination from another source.

34. The method of claim 32 further comprising mapping the taggant of the signature product to the product code or the batch code of the signature product through the use of a decoder key.

35. The method of claim 32 further comprising obtaining the mapped taggant of the signature product to the product code or the batch code of the signature product from a supplier website or database.

36. The method of claim 35 further comprising comparing the mapped taggant of the signature product to the product code or the batch code of the signature product with an immunoassay carried out on a purchased product to determine condition of the purchased product.

37. The method of claim 32 wherein the immunoassay is carried out using a test strip that is specific for the taggant.

38. The method of claim 37 wherein the test strip is a coded test strip that can be read by a bar code reader.

39. The method of claim 37 wherein the test strip comprises a taggant and a product identification.

40. The method of claim 37 wherein the test strip is a lateral flow immunoassay.

41. The method of claim 32 wherein the taggant comprises one or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

42. The method of claim 32 wherein the taggant comprises a taggant array, and wherein the taggant array comprises two or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

43. The method of claim 41 wherein the taggant comprises one or more aliphatic amide compounds or one or more cyclic amide compounds.

44. The method of claim 43 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

45. The method of claim 43 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

46. The method of claim 42 wherein the taggant array comprises (i) two or more aliphatic amide compounds, (ii) two or more cyclic amide compounds, or (iii) a mixture of at least one aliphatic amide compound and at least one cyclic amide compound.

47. The method of claim 46 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

48. The method of claim 46 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7,8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

49. The method of claim 32 wherein the taggant is present in an amount of from 0.05 ppm to 20 ppm.

50. The method of claim 32 wherein the taggant is soluble in water.

51. The method of claim 32 wherein the signature product and the test product are selected from the group consisting of lubricating oils, automatic transmission fluids, engine oils, traction drive transmission fluids, manual transmission fluids, power steering fluids, antifreeze fluids, greases, crankcase lubricants, mineral oils, oils with Group 1, 2, 3 or 4 base oils, differential lubricants, turbine lubricants, gear lubricants, gear box lubricants, axle lubricants, brake fluids, farm tractor fluids, transformer fluids, compressor fluids, cooling system fluids, metal working fluids, hydraulic fluids, industrial fluids, fuels, continuously variable transmission fluid, infinitely variable transmission fluids, and mixtures thereof.

52. The method of claim 32 wherein the signature product and the test product are lubricating oils.

53. A lubricating engine oil having a composition comprising a lubricating oil base stock as a major component; and a taggant, as a minor component; wherein the taggant exhibits degradation in response to one or more stimuli; and wherein the taggant is present in an amount sufficient for an immunoassay to be carried out specific for the taggant to determine degradation of the taggant.

54. The lubricating engine oil of claim 53 wherein the taggant comprises one or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

55. The lubricating engine oil of claim 53 wherein the taggant comprises a taggant array, and wherein the taggant array comprises two or more amide compounds, aminic compounds, aromatic compounds, phenolic compounds, sulfur-containing compounds, heterocyclic compounds, ester compounds, carboxylic acid compounds, aldehyde compounds, ketone compounds, alcohol compounds, imide compounds, acidic compounds, basic compounds, compounds sensitive to oxidation, compounds sensitive to reduction, thermally labile compounds, yellow-metal active compounds, volatile compounds, hydrolytically unstable compounds, surface active compounds, contaminant scavenging compounds, elastomer partitioning additives, oxygen sensitive compounds, light sensitive compounds, or combinations thereof.

56. The lubricating engine oil of claim 54 wherein the taggant comprises one or more aliphatic amide compounds or one or more cyclic amide compounds.

57. The lubricating engine oil of claim 56 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

58. The lubricating engine oil of claim 56 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7, 8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

59. The lubricating engine oil of claim 55 wherein the taggant array comprises (i) two or more aliphatic amide compounds, (ii) two or more cyclic amide compounds, or (iii) a mixture of at least one aliphatic amide compound and at least one cyclic amide compound.

60. The lubricating engine oil of claim 59 wherein the aliphatic amide compounds are selected from pyridine-3-carboxylic acid amide, N-(1-cyano-1,2-dimethylpropyl)-2-(2,4-dichlorophenoxy)propanamide, 1-benzoyl-4-propionylpiperazine, (2S)-2-{[(2S)-2-aminobutanoyl]amino}propanoic acid, 3-[decyl(dimethyl)silyl]-N-[2-(4-methylphenyl)-1-phenylethyl]propanamide, N-allyl-4,5-dimethyl-2-(trimethylsilyl)-3-thiophenecarboxamide, benzyl (1S)-2-({2-[(2-amino-2-oxoethyl)amino]-2-oxoethyl}amino)-1-benzyl-2-oxoethylcarbamate, benzyl (1S,2S)-1-({[(1R)-2-amino-1-benzyl-2-oxoethyl]amino}carbonyl)-2-hydroxypropylcarbamate, N-cyclohexyl-N-methyl-4-[(2-oxo-1,2-dihydro-6-quinolinyl)oxy]butanamide, N-[2-(1H-indol-3-yl)ethyl]tetracosanamide, and N-[2-(1H-indol-3-yl)ethyl]heptadecanamide.

61. The lubricating engine oil of claim 59 wherein the cyclic amide compounds are selected from (5S)-1-methyl-5-(3-pyridyl)pyrrolidin-2-one, 1,3-diethyl-2-thioxodihydro-4,6(1H,5H)-pyrimidinedione, N-[2-(1H-indol-3-yl)ethyl]heptadecanamide, 3-{2-[4-(6-fluoro-1,2-benzisoxazol-3-yl)-1-piperidinyl]ethyl}-2-methyl-6,7, 8,9-tetrahydro-4H-pyrido[1,2-a]pyrimidin-4-one, biotinyl-N-hydroxy-succinimide, 4-ethyl-2-pyrrolidinone, 2-azaspiro[4.6]undecan-3-one, 2-azaspiro[4.4]nonan-3-one, ethyl 2-oxo-3-pyrrolidinecarboxylate, ethyl 5-oxo-3-pyrrolidinecarboxylate, N-(2-ethylhexyl)-5-norbornene-2,3-dicarboximide, 1-(3-aminophenyl)-2-pyrrolidinone, N-(2,6-dimethylphenyl)-2-(2-oxo-1-pyrrolidinyl)acetamide, (3 S)-5-oxo-1-[(1S)-1-phenylethyl]-3-pyrrolidinecarboxylic acid, methyl 2-{[(2,5-dioxo-1-pyrrolidinyl)oxy]carbonyl}benzoate, 4-[3-(cyclopentyloxy)-4-methoxyphenyl]-2-pyrrolidinone, 1-{[(7-hydroxy-2-oxo-2H-chromen-3-yl)carbonyl]oxy}-2,5-pyrrolidinedione, 2-(1-hydroxyundecyl)-1-(4-nitroanilino)-6-phenyl-4a,7a-dihydro-1H-pyrrolo[3,4-b]pyridine-5,7(2H,6H)-dione, 1-methyl-2-piperidinone, 1,5-dimethyl-2-piperidinone, 1,4,4-trimethyl-2,6-piperidinedione, 4-methyl-1-undecyl-2-piperidinone, 4-methyl-1-decyl-2-piperidinone, 1-(1-adamantyl)-2-piperidinone, 3,3-(butane-1,4-diyl)bis(1,8,8-trimethyl-3-azabicyclo[3.2.1]octane-2,4-dione), and tert-butyl 2,4-dioxo-1-piperidinecarboxylate.

62. The lubricating engine oil of claim 53 wherein the taggant is present in an amount of from 0.05 ppm to 20 ppm.

63. The lubricating engine oil of claim 53 wherein the lubricating oil base stock comprises a Group I, Group II, Group III, Group IV, or Group V base oil.

64. The lubricating engine oil of claim 53 wherein the lubricating oil base stock is present in an amount of from 70 weight percent to 95 weight percent, based on the total weight of the lubricating engine oil.

65. The lubricating engine oil of claim 53 further comprising one or more of an antiwear additive, viscosity modifiers, antioxidant, detergent, dispersant, pour point depressant, corrosion inhibitor, metal deactivator, seal compatibility additive, anti-foam agent, inhibitor, and anti-rust additive.

66. The lubricating engine oil of claim 53 which is a passenger vehicle engine oil (PVEO).

67. A diagnostic kit for the analysis of products, said kit comprising one or more immunoassay interrogation devices, one or more immunoassay test strips, and immunoassay instructions.

68. The diagnostic kit of claim 67 wherein the products comprise lubricating oils.
